SEQUENCE LISTING (24 sequences)

SEQ ID NO: 1 - protein, 15 aa, Human immunodeficiency virus
RIQRGPGRAFVTIGK

SEQ ID NO: 2 - protein, 13 aa, Human immunodeficiency virus
SVITQACSKVSFE

SEQ ID NO: 3 - protein, 13 aa, Human immunodeficiency virus
GTGPCTNVSTVQC

SEQ ID NO: 4 - protein, 13 aa, Human immunodeficiency virus
LWDQSLKPCVKLT

SEQ ID NO: 5 - protein, 11 aa, Human immunodeficiency virus
VYYGVPVWKEA

SEQ ID NO: 6 - protein, 12 aa, Human immunodeficiency virus
YLRDQQLLGIWG

SEQ ID NO: 7 - protein, 25 aa, Human immunodeficiency virus
FLGFLGAAGSTMGAASLTLTVQARQ

SEQ ID NO: 8 - protein, 15 aa, Human immunodeficiency virus
IFPGKRTIVAGQRGR

SEQ ID NO: 9 - protein, 13 aa, Influenza virus
PRYVKQNTLKLAT

SEQ ID NO: 10 - protein, 13 aa, Influenza virus
PKYVKSNRLVLAT

SEQ ID NO: 11 - protein, 514 aa, Influenza virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEEHHHHHH

SEQ ID NO: 12 - protein, 517 aa, Influenza virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEHHHHHH

SEQ ID NO: 13 - protein, 451 aa, Artificial sequence
  Synthetic construct
HMSLLTEVETPIRNEWGCRCNDSSDSHSIQTGSQNHTGICNQNIITYKNSTWVKDTTSVILTGNSSLCPIRGWAIYSKDNSIRIGSKGDVFVIREPFISCSHLECSTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDKHHHHHH

SEQ ID NO: 14 - protein, 446 aa, Artificial sequence
  Synthetic construct
HMSLLTEVETPTRNEWECRCSDSSDSHSIHTGNQHQSEPISNTNFLTEKAVASVKLAGNSSLCPINGWAVYSKDNSIRIGSKGDVFVIREPFISCSHLECSTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDKHHHHHH

SEQ ID NO: 15 - protein, 407 aa, Artificial sequence
  Synthetic construct
MSHSIQTGSQNHTGICNQNIITYKNSTWVKDTTSVILTGNSSLCPIRGWAIYSKDNSIRIGSKGDVFVIREPFISCSHLECSTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFSIDKHHHHHH

SEQ ID NO: 16 - protein, 503 aa, Artificial sequence
  Synthetic construct
MASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEETSHHHHHH

SEQ ID NO: 17 - protein, 254 aa, Artificial sequence
  Synthetic construct
MFQDPQERPRKLPQLCTELQTTIHDIILECVYCKQQLLRREVGDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPHHHHHH

SEQ ID NO: 18 - protein, 427 aa, Artificial sequence
  Synthetic construct
MSHSIQTGSQNHTGICNQNIITYKNSTWVKDTTSVILTGNSSLCPIRGWAIYSKDNSIRIGSKGDVFVIREPFISCSHLECSTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDKHHHHHH

SEQ ID NO: 19 - protein, 422 aa, Artificial sequence
  Synthetic construct
MSHSIHTGNQHQSEPISNTNFLTEKAVASVKLAGNSSLCPINGWAVYSKDNSIRIGSKGDVFVIREPFISCSHLECSTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDKHHHHHH

SEQ ID NO: 20 - DNA, 20 bp, Artificial sequence
  Synthetic construct
tccatgacgttcctgatgct

SEQ ID NO: 21 - protein, 9 aa, Homo sapiens
AAGIGILTV

SEQ ID NO: 22 - protein, 9 aa, Homo sapiens
ITDQVPFSV

SEQ ID NO: 23 - protein, 9 aa, Homo sapiens
KTWGQYWQV

SEQ ID NO: 24 - protein, 9 aa, Homo sapiens
YLEPGPVTA

(19) United States
(12) Patent Application Publication
(10) Pub. No.: US 2007/0160622 A1
(43) Pub. Date: Jul. 12, 2007

(54) Method for assembling a polymer-biologic delivery composition

(75) Inventors: William G. Turnell, Del Mar, CA (US); Benjamin W. Parcher, San Diego, CA (US); Catherine H. Charles, Encinitas, CA (US); Chittari Pabba, San Diego, CA (US); Maria A. Vitiello, La Jolla, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/636,230

(22) Filed: Dec. 7, 2006

Related U.S. Application Data
(60) Provisional application No. 60/748,486, filed on Dec. 7, 2005.
(60) Provisional application No. 60/858,173, filed on Nov. 10, 2006.

(51) Int. Cl. A61K 39/145; A61K 39/00; A61K 47/48; C08F 20/00; C08G 63/91

(52) U.S. Cl. 424/185.1; 525/54.1; 525/440; 424/78.27; 424/209.1

(57) ABSTRACT

A one-step method for assembly of delivery compositions for one or more antigens or therapeutic biologics is based on non-covalent affinity capture of molecules from solution using a biodegradable polymer having functional groups to which the affinity ligand binds. The polymer-bound affinity complex, which includes the molecule(s) of interest is then recovered from the reaction solution, for example, by size exclusion filtration, to yield the assembled delivery composition. The affinity ligand can be a monoclonal antibody or a metal affinity ligand with bound metal transition ion. The assembled delivery compositions can be formulated as polymer particles, which can then be lyophilized and reconstituted for in vivo delivery of the non-covalently complexed antigen(s) or therapeutic biologic(s) with substantial native activity.

### RELATED APPLICATIONS

This application relies for priority under 35 U.S.C. § 119(e) on provisional application 60/748,486, filed Dec. 7, 2005.

### FIELD OF THE INVENTION

The invention relates generally to method for preparation of polymer-based delivery compositions and, in particular, to methods for assembly of polymer-based vaccines and delivery compositions for biologics.

### BACKGROUND INFORMATION

Although significant progress in vaccine development and administration has been made, alternative approaches that enhance the efficacy and safety of vaccine preparations remain under investigation. Synthetic vaccines, so called because of the use of defined antigens such as recombinant proteins, synthetic peptides, and polysaccharide-peptide conjugates, are emerging as novel vaccine candidates. Traditional vaccines are made of attenuated or inactivated pathogens, or purified bacterial or viral components. Synthetic vaccines represent a safe and flexible alternative to traditional vaccines, but further effort is required to increase the immunogenicity, and thus the efficacy, of these vaccines. To induce an effective immune response, a specific antigen, such as a viral protein or peptide, must be presented to the immune system in an immunogenic form. Materials and substances that potentiate an immune response to a specific antigen are known as “adjuvants”. Known adjuvants either facilitate the delivery of antigen to the specialized cells that activate the immune system, or directly stimulate and induce maturation of these cells. These two functions effectively mimic the stimulatory effects of natural pathogens on the immune system. Synthetic vaccines, therefore, will need to deliver antigens in an immunostimulatory way.

Currently, aluminum compounds remain the only FDA approved adjuvants for use in human vaccines in the United States. Despite their good safety record, aluminum compounds are relatively weak adjuvants and often require multiple dose regimens to elicit antibody levels associated with protective immunity. In addition, aluminum compounds are not effective in generating cell-mediated immunity and, therefore, may not be ideal adjuvants for situations in which a cell-mediated response is required, as is thought to be the case for many viral infections, chronic infections, and malignancies. Although many candidate adjuvants are presently under investigation, a number of disadvantages, including toxicity in humans and requirements for sophisticated techniques to incorporate antigens remain to be overcome.

An efficacious vaccine should induce a protective or therapeutic immune response as required to neutralize an infection or destroy aberrant cells (infected or transformed). The adaptive immune response, i.e., the antigen specific response is mediated by lymphocytes and in particular by T and B lymphocytes. B lymphocytes recognize and bind antigens using their membrane antigen-specific receptors: the antibody molecules. Each B cell expresses a unique antibody receptor that will be secreted after B cell stimulation and will bind to the antigen with the intent of ridding the organism of the antigen. The antibody response is useful, for example, for neutralization of viruses. In this case it is important that the antibody recognizes the same viral epitopes used by the virus to enter, infect, or damage a cell. In this case, it is necessary that the antigen used for vaccine preparation have the same conformation as the antigen in the virus. On the other hand, T lymphocytes do not recognize free antigen, but only antigen in the context of MHC molecules. There are two main classes of MHC molecules. Class I molecules are synthesized and displayed by most of the cells of the body, while Class II molecules are presented almost exclusively by antigen presenting cells (APC). T cells with the CD4 phenotype, also called helper T cells, recognize antigens in the context of MHC Class II proteins and, upon activation, secrete lymphokines and directly activate the cells with which they are interacting. On the other hand, T cells with the CD8 phenotype recognize antigens in the context of MHC Class I proteins. Upon activation, T cells secrete lymphokines and can kill the cell they recognize.

Exogenous antigens are immunogenic materials not normally present in the host organism. Examples are derived from bacteria, free viruses, yeasts, protozoa, and toxins. These exogenous antigens enter antigen-presenting cells or APCs (macrophages, dendritic cells, and B-lymphocytes) through phagocytosis, macropinocytosis or by receptor mediated uptake. The microbes are engulfed and protein antigens are degraded by proteases into a series of peptides. These peptides eventually bind to a groove in MHC molecules and are transported to the surface of the APC. CD4-lymphocytes are then able to recognize peptide/MHC-II complexes by means of their T cell receptors (TCRs) and CD4 molecules. Peptides that are presented by APCs in class II MHCs are about 10 to about 30 amino acids, for example about 12 to about 24 amino acids in length (Marsh, S. G. E. et al. (2000) The HLA Facts Book, Academic Press, p. 58-59). The effector functions CD4-lymphocytes include activating B cells for maturation, class switching and antibody production. CD4 T cells also activate dendritic cells (DC) to secrete cytokines and stimulate cytotoxic T cells, and increase microbiocidal activities of macrophages, all of which are important mechanisms by which extracellular or intracellular pathogens are destroyed. CD8-lymphocytes are able to recognize peptide/MHC-I complexes by means of their T cell receptors (TCRs) and CD8 molecules. Peptides that are presented by APCs in class I MHCs are about 8 to about 17 amino acids in length.

One of the body's major defenses against viruses, intracellular bacteria, and cancers is destruction of endogenous infected cells and tumor cells by cytotoxic T-lymphocytes or CTLs. These CTLs are effector cells derived from CD8 positive T-lymphocytes (CD8 T cells). In order to become CTLs, naive CD8 T cells must become activated by APCs. The process involves dendritic cells engulfing and degrading infected cells, tumor cells, and the remains of killed infected and tumor cells. It is thought that in this manner, endogenous antigens from diseased cells are able to enter the APC, where proteases and peptidases degrade the protein into a series of peptides, of about 8 to about 10, possibly about 8 to about 11, or about 8 to about 12 amino acids in length. The MHC class I molecules with bound peptide, which appear on the surface of the APCs, can now be recognized by naive CD8 T cells possessing T cell receptors (TCRs) with a complementary binding surface. This recognition of the peptide epitope by the TCR serves as a first signal for activating the naive CD8 T cell and inducing effector (CTL) function. Complete activation of T cells requires a second, non-antigen specific signal, most often provided by the same cognate APC. These second signals are often provided by molecules upregulated by an APC in response to immunostimulatory adjuvants, such as Toll-Like Receptor (TLR) agonists.

An additional area of interest in the drive to prepare synthetic vaccines is development of methods for rapid purification of recombinant proteins. A number of methods have been developed based on specific interactions between an affinity tag and an immobilized ligand. The most widely used of these is immobilized metal-affinity chromatography (IMAC), which employs the principle of selective interaction between a solid matrix containing immobilized metal ions such as Cu²⁺ or Ni²⁺ and a poly-histidine tag fused to the protein. Proteins containing a polyhistidine tag are selectively bound to the matrix while other proteins are washed away.

Metal-affinity precipitation, an alternative to IMAC, does not employ an immobilized ligand. Instead, target poly-histidine-tagged recombinant proteins bind specifically to polymer-metal ligand conjugates that precipitate from solution in response to an environmental trigger, such as pH or temperature. This phenomenon allows purification of the recombinant protein from other cell extracts by precipitation. The purified proteins are recovered by dissociation from the polymer conjugates, which can be recycled for subsequent reuse. Poly(N-isopropylacrylamide) and recombinant elastin-like proteins, the latter having a valine residue at the fourth position in elastin monomers replaced with a lysine, have been used to create the required metal coordination chemistry for metal-affinity precipitation. However, neither method is straightforward. For example, the elastin-like polymers themselves require recombinant preparation.

A related problem is preparation of compositions for in vivo delivery of various therapeutic biologics, such as polynucleotides, proteins and the like without destruction of native activity of the molecules.

Thus, there is still a need in the art for new and better methods for preparing vaccine delivery compositions utilizing protein and other antigens and adjuvants in the place of deactivated pathogens. There is also a need in the art for new and improved methods for assembling, from solution or dispersion, compositions for in vivo delivery of therapeutic biologics with substantial native activity.

### SUMMARY OF THE INVENTION

The present invention adapts a metal-affinity purification technique to create a one-step method for assembly from solution or dispersion of compositions for delivery of therapeutic biologics and vaccines using a biodegradable polymer. Biodegradable polymers that contain functional groups on the polymer molecules can be used to capture from a solution or dispersion at least one therapeutic biologic or antigen (with or without the presence of an adjuvant) in a one-step assembly procedure. For example, in the invention one-step vaccine assembly method, polymers that contain amino acids in the polymer chain, such as certain poly(ester amide) (PEA), poly(ester urethane) (PEUR), and poly(ester urea) (PEU) polymers, can be used in one-step assembly of synthetic and, hence, easy to produce vaccine delivery compositions by specifically capturing one or more antigens in an affinity complex that forms as an attachment to the polymer. Although the invention methods are illustrated herein with reference to formation of vaccine delivery compositions with immunogenic and therapeutic utility, the methods described herein can also be used for one-step assembly of compositions for in vivo delivery of a variety of therapeutic biologics so as to substantially retain the native activity and, hence, therapeutic utility of the biologic molecule(s).

Accordingly, in one embodiment the invention provides methods for assembling a vaccine delivery composition by contacting together in a solution or dispersion a purified molecule containing at least one synthetic antigen, an affinity ligand that binds specifically to the purified molecule, and a synthetic biodegradable polymer containing functional groups to which the affinity ligand can attach. The contacting is conducted under conditions such that the affinity ligand attaches to the polymer via the free functional group(s) and a non-covalent complex forms between the molecule containing the antigen and the polymer-attached specifically binding affinity ligand so as to assemble the vaccine delivery composition in a single step.

In another embodiment, the invention provides methods for assembling a delivery composition for in vivo delivery of a therapeutic biologic by contacting together in a solution or dispersion 1) a purified synthetic molecule in which a therapeutic biologic is attached to a metal-binding amino acid tag, 2) at least one transition metal ion, 3) a metal affinity ligand that binds to the metal ion, and 4) a synthetic biodegradable polymer containing functional groups to which the affinity ligand can attach. The contacting is conducted under conditions such that the affinity ligand attaches to the polymer via the free functional group(s) thereon and a non-covalent complex forms between the polymer-attached metal affinity ligand, the transition metal ion, and the metal binding tag in the synthetic molecule so as to assemble the composition while maintaining substantial native activity of the biologic.

In yet another embodiment, the invention provides compositions suitable for use in the invention assembly methods. The invention compositions contain a synthetic biodegradable polymer having one or more functional groups to which is preattached a metal affinity ligand that has been non-covalently complexed with a transition metal ion, wherein the composition is soluble.

In still another embodiment, the invention provides methods for delivering a vaccine or therapeutic biologic to a subject by administering to the subject an invention vaccine delivery or therapeutic biologic delivery composition made by the invention methods.

In yet another embodiment, the invention provides compositions in which a synthetic biodegradable polymer is attached via a functional group thereon to a metal affinity ligand, which is non-covalently complexed with a metal transition ion, wherein the composition is soluble in aqueous media.

### BRIEF DESCRIPTION OF THE FIGURES

FIG. 1 is a graph showing tumor mass in tumors excised from mice challenged with C3, a human papilloma virus (HPV)-expressing tumor cell line, 5 weeks after a single injection with the indicated compositions admixed with CpG as adjuvant (5 nmol per mouse) prior to immunization. Mice injected with irradiated cells and untreated mice are control groups. Tumor size was assessed 15 days after tumor cell challenge. Each symbol indicates the mass of tumor from an individual animal.

FIG. 2 is a graph showing tumor size in mice challenged with C3, an HPV-expressing tumor cell line, one week after a single immunization with the indicated composition without additional adjuvant. Tumor size was assessed on day 18 post-challenge. Each symbol indicates the relative tumor size from an individual animal. Bars represent average tumor size for each group of mice.

FIG. 3 is a graph showing tumor size in mice injected with C3, an HPV-expressing tumor cell line. Six days after cell injection, the mice received a single, subcutaneous injection with the indicated composition. Tumor size was assessed over 24 days following tumor cell injection. Each symbol indicates the relative tumor size from an individual animal. Bars represent average tumor size for each group of mice.

FIG. 4 is a graph showing anti-HA titer (primary response) after mice received a single injection and were boosted with the indicated formulations, with or without PEA polymer in the formulation, PBS (negative control) or infectious PR8 virus (positive control). Serum samples were collected 20 days after the first injection and 14 days after immunization.

FIG. 5 is a graph showing secondary anti-HA IgG2a response after a single injection with the indicated formulations, with or without PEA polymer in the formulation. Animal groups receiving PBS (negative control) or infectious PR8 virus (positive control) are included for comparison. Mice were primed and boosted 21 days later with the indicated formulations. Serum samples were collected 14 days after the boost and secondary response anti-HA IgG2a titers determined by ELISA.

FIG. 6 is a graph showing viral neutralization serum titers in mice injected and boosted with the indicated formulations and controls as in FIGS. 4 and 5. Serum samples were collected 20 days after the first injection and 14 days after the boost. Serum neutralizing titers against HA were determined by an influenza virus microneutralization assay using MDCK cells. After the boost, all formulations that included HA induced measurable levels of neutralizing antibodies

FIG. 7 is a graph showing weight change after challenge with infectious virus in mice injected and boosted with the indicated vaccine formulations of FIGS. 4, 5 and 6. Mice were challenged intranasally with infectious PR8 virus. Dotted line at −20% represents the point at which animals had to be euthanized.

FIG. 8 is a graph showing survival of the mice after infectious challenge. Mice were injected and boosted intraperitoneally (ip) with the indicated vaccine formulations. Mice were challenged intranasally with infectious PR8 virus and euthanized according to protocol, when weight loss was 20% or more.

FIG. 9 is a graph showing antibody response in study mice injected ip with the indicated formulations based on influgenza.A/Vietnam/1203/2004H₅N1 molecules. Serum samples were collected 12 days later and IgG1 titers determined by end-point ELISA. Data is reported as the reciprocal of the serum dilution that gives a reading 2 standard deviations above background.

FIG. 10 is a graph showing survival of immunized study ferrets after infectious intranasal challenge by 1.3×10³ TCID₅₀ of A/Vietnam/1203/2004 influenza virus. Ferrets were injected and boosted with the indicated viral antigens complexed with PEA polymer. Ferrets were euthanized 20 days after challenge according to protocol.

FIG. 11 is a graph showing weight loss in study ferrets after infectious challenge with Influenza A/Vietnam/1203/2004 as in FIG. 10: Ferrets were injected and boosted with the indicated viral antigens complexed with PEA polymer, or with PBS as the negative control. Weight change in study ferrets was monitored for 20 days after intranasal challenge with infectious virus.

FIGS. 12A-D are a set of graphs showing hematological data collected from blood drawn from study ferrets 3 days after infectious intranasal challenge with Vietnam Influenza A virus. The ferrets had been injected and boosted with the indicated viral antigens complexed with PEA polymer. Ferrets were challenged intranasally and bled 3 days after challenge. Dotted lines represent normal ranges. FIG. 12A=white blood cells (WBC), FIG. 12B=lymphocytes, FIG. 12C=monocytes, and FIG. 12D=platelets (PLT) in the virus challenged ferrets.

FIG. 13 is the amino acid sequence in single letter code for the expressed ectodomain of hemagglutinin protein from A/Puerto Rico/8/34 (H1N1) (SEQ ID NO:11).

FIG. 14 is the amino acid sequence in single letter code for the expressed ectodomain of hemagglutinin protein from A/Vietnam/1203/2004 (H₅N₁) (SEQ ID NO:12).

FIG. 15 is the amino acid sequence in single letter code for the fusion protein of the ectodomain of the M2 protein and the ectodomain of neuraminidase derived from A/Puerto Rico/8/34 (H1N1) (SEQ ID NO:13).

FIG. 16 is the amino acid sequence in single letter code for the fusion protein of the ectodomain of the M2 protein and the ectodomain of neuraminidase derived from A/Vietnam/1203/2004 (H₅N1) (SEQ ID NO: 14).

FIG. 17 is the amino acid sequence in single letter code for His-tagged version of nucleoprotein derived from A/Puerto Rico/8/34 (H1N1) (SEQ ID NO:15).

FIG. 18 is the amino acid sequence in single letter code for His-tagged version of nucleoprotein derived from A/Vietnam/1203/2004 (H₅N1) (SEQ ID NO:16).

FIG. 19 is the amino acid sequence in single letter code for the expressed mutated fusion protein of HPV-16 E6 and E7. The amino terminal underlined sequence is from E6; the central portion is from E7 and there is a carboxy-terminal hexa-histidine tag (SEQ ID NO:17).

FIG. 20 is the amino acid sequence in single letter code for the ectodomain of neuraminidase derived from A/Puerto Rico/8/34 (H1N1) (SEQ ID NO:18).

FIG. 21 is the amino acid sequence in single letter code for the ectodomain of neuraminidase derived from A/Vietnam/1203/2004 (H₅N₁) (SEQ ID NO:19).

### DETAILED DESCRIPTION OF THE INVENTION

The invention is based on the discovery that under the right conditions biodegradable polymers that contain functional groups on the polymer molecules can be used to capture purified target molecules, such as at least one antigen, from a dispersion, cell lysate, or solution while non-covalently binding the captured molecule to the polymer by means of an affinity ligand that binds specifically to sites on the target molecule. The type of affinity ligand attached to the functional groups on the polymer depends upon the characteristics of the target molecule. For example, a target molecule in solution, such as a protein, fusion protein, or other molecule that is engineered to contain (or naturally contains) metal-binding amino acids will bind specifically, yet non-covalently, with a metal affinity ligand and metal ion bound to the polymer to capture the target molecule in a metal affinity complex. Target molecules that contain a specific antibody binding site can be similarly captured by a monoclonal antibody conjugated to the polymer. This discovery is used in the present invention for one-step assembly of a polymer-based delivery composition.

The polymers preferred for use in the invention methods, the PEAs, PEURs and PEUs described by structural formulas (I and III-VII), not only contain the functional groups used in the invention methods, but also have delivery-adjuvant activity and are readily taken up by antigen presenting cells (APCs). Thus these polymers both facilitate the invention methods for assembly of delivery compositions, but are especially suited for vaccine assembly and enhance the immunogenicity of the vaccine delivery compositions made by the invention methods.

Accordingly, in one embodiment of the invention methods comprise contacting the following elements together in a solution or dispersion: 1) a purified molecule containing at least one synthetic antigen; 2) an affinity ligand that binds specifically to the purified molecule; and 3) a synthetic biodegradable polymer containing functional groups to which the affinity ligand can conjugate or has been preattached. The contacting is conducted under conditions such that the functional groups on the polymer attach to the affinity ligand and a non-covalent affinity complex forms containing the antigen so as to assemble the vaccine delivery composition in a single step.

In one embodiment, synthetic molecules that include one or more antigens or therapeutic biologics of interest and which are engineered to add an amino-acid containing tag, such as a hexaHistidine tag, are readily assembled from solution into a polymer-based delivery composition according to the invention methods. A metal affinity complex forms to non-covalently link the molecule containing the at least one antigen or therapeutic biologic to a biodegradable polymer. Polymers used in the invention methods have free functional groups to which the affinity ligand is conjugated. For example, polymers that contain amino acids in the polymer chain, such as those that contain at least one amino acid conjugated to at least one non-amino acid moiety per monomer, can be used to prepare synthetic and, hence, easy to produce polymer-based compositions for in vivo delivery of at least one antigen or therapeutic biologic with substantial native activity. Hence, the invention delivery compositions possess utility for in vivo delivery of biologics for treatment of various diseases and for stimulating an immune response to a variety of pathogenic organisms or malignancies in humans and other animals.

In the invention methods, such biodegradable polymers are used to prepare a synthetic delivery composition for subcutaneous or intramuscular injection or mucosal administration. The compositions are reproducible in large quantities using the invention methods, safe (the vaccine delivery compositions contain no attenuated pathogen), stable, and can be lyophilized for transportation and storage. Due to structural properties of the polymer used, the delivery compositions assembled by the invention methods provide high copy number and local density of antigen or therapeutic biologic.

For example, in one embodiment, the invention provides methods for assembly of a vaccine delivery composition by contacting together in a solution or dispersion 1) a lysate or extract of an organism that contains at least one recombinant vector comprising a vector and a DNA sequence insert that encodes a protein antigen that contains at least one Class I or Class II restricted epitope comprising from 5 to about 30 amino acids, wherein the antigen has been expressed by the organism; 2) a transition metal ion selected from Cu²⁺, Ni²⁺, Co²⁺, and Zn²⁺ ions; 3) a metal affinity ligand that binds to the metal ion; and 4) a synthetic biodegradable polymer with free functional groups. These elements are contacted under conditions such that the free functional groups on the polymer bind to the metal affinity ligand and a non-covalent complex is formed that incorporates the polymer-attached metal affinity ligand, the transition metal ion, and the at least one antigen. Optionally, but preferably, the metal affinity ligand and metal ion can be preattached to the functional groups on the polymer, as described herein, prior to introducing the polymer into the solution or dispersion containing the target molecule.

In yet another embodiment, the polymer with attached affinity ligand and metal ion can be formulated as a polymer particle, for example as described herein prior to contacting the solution or dispersion containing the purified molecule containing the antigen or therapeutic biologic. The invention method can further comprise separating the affinity complex and bound polymer or particles thereof, from the solution or dispersion to obtain the composition free of undesired components, for example, by size exclusion technology.

The invention delivery composition so prepared can be formulated to achieve compositions with different properties. In one embodiment, the polymer acts as a time-release polymer depot releasing antigen and antigen-polymer fragments to be taken up by APCs and presented by MHC class I or class II molecules as the polymer depot biodegrades in vivo. In other embodiments, the polymer acts as a carrier for the antigen into the APC, and the antigen is degraded enzymatically for presentation on the cell surface in the context of MHC class I or class II molecules. In another embodiment, the polymer acts to protect an antigen and facilitate its delivery to a local lymph node, where antigen-specific B lymphocytes can recognize an antigen that is presented in native conformation. The presence of the polymer, metal transition ion and affinity ligand in the composition do not interfere with these biological processes.

In addition to treatment of humans, delivery compositions produced by the invention methods are also intended for use in veterinary treatment of a variety of animal patients, such as pets (for example, cats, dogs, rabbits, and ferrets), farm animals (for example, chicken, ducks, swine, horses, mules, dairy and meat cattle) and race horses.

Invention methods and vaccine delivery compositions can utilize protein or protein subunit antigens, or other types of antigens, which are non-covalently attached to the polymer via metal affinity complexes formed at functional groups on the polymer molecules. Optionally, immunostimulatory adjuvants may be dispersed in or attached to the polymer as well. APCs display antigen-derived peptides via MHC complexes and are recognized by T cells, such as cytotoxic T cells, to generate and promote endogenous immune responses leading to destruction of pathogenic cells bearing matching or similar antigens. Alternatively, APCs can present unprocessed, whole protein antigen on their surfaces, which can then be recognized by antigen-specific B cells. The polymers used in the invention vaccine delivery composition can be designed to tailor the rate of biodegradation of polymer molecules or depots and particles formulated thereof to result in sustained availability of antigen-APC complexes over a sustained period of time. For instance, typically, the polymer depot will degrade over a time ranging from about twenty-four hours, about seven days, about thirty days, or about ninety days, or longer, depending upon selection of the monomers used in fabrication of the delivery polymer. Longer time spans are particularly suitable for providing an implantable vaccine delivery composition that eliminates the need to repeatedly inject the vaccine to obtain a suitable immune response.

The vaccine delivery compositions prepared by the invention methods utilize biodegradable polymer-mediated delivery techniques to elicit an immune response against a wide variety of pathogens, including mucosally transmitted pathogens. The compositions afford a vigorous immune response, even when the antigen is by itself weakly immunogenic. Although the individual components of the vaccine delivery composition and methods of preparation thereof described herein were known, it was unexpected and surprising that such methods and combinations of active agents would enhance the efficiency of antigens beyond levels achieved when the components were used separately and, moreover, that the polymers used in making the vaccine delivery composition may obviate the need for additional adjuvants in some cases while reducing the technique of purifying recombinant antigens and fabricating polymer-containing vaccines to a one-step method.

Although the invention is broadly applicable to providing vaccine delivery compositions for providing an immune response against any of the above-mentioned pathogens, the invention is exemplified herein by reference to influenza virus and HPV.

The vaccine delivery compositions, as prepared by the methods of the invention, provide for cell-mediated immunity, and/or humoral antibody responses. Accordingly, the methods of the present invention will find use with any antigen for which cellular and/or humoral immune responses are desired, including antigens derived from viral, bacterial, fungal and parasitic pathogens as well as tumor associated antigens that may induce antibodies, T-helper cell activity and T cell cytotoxic activity. Thus, “immune response” as used herein means production of antibodies, T-helper cell activity or T cell cytotoxic activity specific to the antigen used. Such antigens include, but are not limited to those encoded by human and animal pathogens and can correspond to either structural or non-structural proteins, polysaccharide-peptide conjugates, RNA or DNA.

For example, the present invention will find use in preparation of vaccine delivery compositions for stimulating an immune response against a wide variety of proteins from the herpes virus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpes viruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV.)

Antigens from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Each of these proteins, as well as antigenic fragments thereof, will find use in the present methods. Similarly, the sequence for the 6-antigen from HDV is known (see, e.g., U.S. Pat. No. 5,378,814) and this antigen can also be conveniently used in the present methods. Additionally, antigens derived from HBV, such as the core antigen, the surface antigen, sAg, as well as the presurface sequences, pre-S1 and pre-S2 (formerly called pre-S), as well as combinations of the above, such as sAg/pre-S1, sAg/pre-S2, sAg/pre-S1/pre-S2, and pre-S1/pre-S2, will find use herein. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513, incorporated herein by reference in their entireties; Beames et al., J. Virol. (1995) 69:6833-6838, Birnbaum et al. J. Virol. (1990) 64:3319-3330; and Zhou et al. J. Virol. (1991) 65:5457-5464.

Antigens derived from other viruses will also find use in the claimed methods, such as without limitation, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviddae; Arenaviridae; Retroviradae (e.g., HTLV-I; HTLV-II; HIV-1 (also known as HTLV-III LAV, ARV, hTLR, etc.)), including but not limited to antigens from the isolates HIV_{IIIb}, HIV_{SF2}, HIV_{LAV}, HIV_{LA1}, HIV_{MN}); HIV-1_{CM235}, HIV-1_{US4}; HIV-2; simian immunodeficiency virus (SIV) among others. Additionally, antigens may also be derived from HPV and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses.

More particularly, the envelope proteins from any of the above HIV isolates, including members of the various genetic subtypes of HIV, are known and reported (see, e.g., Myers et al., Los Alamos Database, Los Alamos National Laboratory, Los Alamos, N.Mex. (1992); Myers et al., Human Retroviruses and Aids, 1990, Los Alamos, N.Mex.: Los Alamos National Laboratory; and Modrow et al., J. Virol. (1987) 61:570-578, for a comparison of the envelope sequences of a variety of HIV isolates) and antigens derived from any of these isolates will find use in the present methods. Specifically, the synthetic peptide, RISK (Nehete et al. Antiviral Res. (2002) 56:233-251), derived from the V3 loop of gp120 and having the sequence RIQRGPGRAFVTIGK (SEQ ID NO:1), will have use in the invention compositions and methods. Furthermore, the invention is equally applicable to other immunogenic proteins derived from any of the various HIV isolates, including any of the various envelope proteins such as gp160 and gp41, gag antigens such as p24gag and p55gag, as well as proteins derived from the pol region. Furthermore, multi-epitope cocktails of polymer-peptide conjugates can be envisioned using various epitopes from HIV proteins. For example, 6 conserved peptides from gp120 and gp41 have been shown to reduce viral load and prevent transmission in a rhesus/SHIV model: SVITQACSKVSFE (S13E) (SEQ ID NO:2), GTGPCTNVSTVQC (G13C) (SEQ ID NO:3), LWDQSLKPCVKLT (L13T) (SEQ ID NO:4), VYYGVPVWKEA (V11A) (SEQ ID NO: 5), YLRDQQLLGIWG (V12G) (SEQ ID NO:6), and FLGFLGAAGSTMGAASLTLTVQARQ (F25Q) (SEQ ID NO:7) (Nehete et al. Vaccine (2001) 20:813-). The amino acid sequence of the antigen tested in the invention compositions and methods is IFPGKRTIVAGQRGR (SEQ ID NO:8), wherein all amino acids are natural, L-amino acids.

As explained above, influenza virus is another example of a virus for which the present invention will be particularly useful. Specifically, the envelope glycoproteins HA and NA of influenza A are of particular interest for generating an immune response, as are the nuclear proteins and can be used to generate vaccine delivery compositions according to the invention methods. Numerous HA subtypes of influenza A have been identified (Kawaoka et al., Virology (1990) 12:759-767; Webster et al., “Antigenic variation among type A influenza viruses,” p. 127-168. In: P. Palese and D. W. Kingsbury (ed.), Genetics of influenza viruses. Springer-Verlag, New York). Thus, proteins derived from any of these isolates can also be used in the immunization techniques described herein. In particular, the conserved 13 amino acid sequence of HA can be used in the invention vaccine delivery composition and methods. In H3 strains used in current vaccine formulations, this amino acid sequence is PRYVKQNTLKLAT (SEQ ID NO:9), and in H5 strains it is predominantly PKYVKSNRLVLAT (SEQ ID NO: 10).

T cell epitopes are small peptides that are contained within a whole antigenic protein as short segments of the amino acid sequence. In vivo, following entry of a protein into an intracellular antigen processing pathway, the protein is cleaved by enzymes so as to liberate the T cell epitopes contained therein for presentation on the surface of antigen presenting cells. In this way, whole proteins or peptides can be delivered as antigens, and the cellular response is to process the whole protein so as to trigger an immune response.

B cell epitopes are conformational determinants that may consist of protein, glycoprotein, lipid or other biological entities. B cells typically recognize unprocessed antigens, such as proteins, on the surface of a pathogen, or on the surface of an antigen presenting cell. B cells typically encounter their cognate antigen in a lymph node or other lymph tissue, where the antigen has been trafficked by an antigen presenting cell. Once activated, the B cell becomes an effector cell, secreting antibodies specific for the antigen, and binding directly to pathogens that carry this antigen on their surfaces. B cells and the antibody response can eliminate or neutralize pathogens by one of several methods. Bacteria or viruses that become coated with secreted antibody are marked for destruction by Fc-receptor carrying cells of the innate immune system. Alternatively, pathogens can be taken up by antigen-specific B cells through receptor mediated endocytosis. These B cells can then act as antigen presenting cells for CD4 T cells, further strengthening the immune response to the pathogen. Another method by which antibodies protect the host is simply through steric interference, such that an antibody-coated pathogen is physically unable to enter a host T cell, or otherwise exert its pathogenic effects. This is known as “neutralization” of a pathogen, and is the basis for critical methods of in vitro analysis of the worth of a vaccine; the vaccine must induce antibodies that are not only specific, but also functionally neutralizing.

In another embodiment of the invention vaccine delivery composition, whole protein structural domains, derived and modified from native viral coat proteins, can be conjugated to PEA, PEUR or PEU polymers and delivered as antigens.

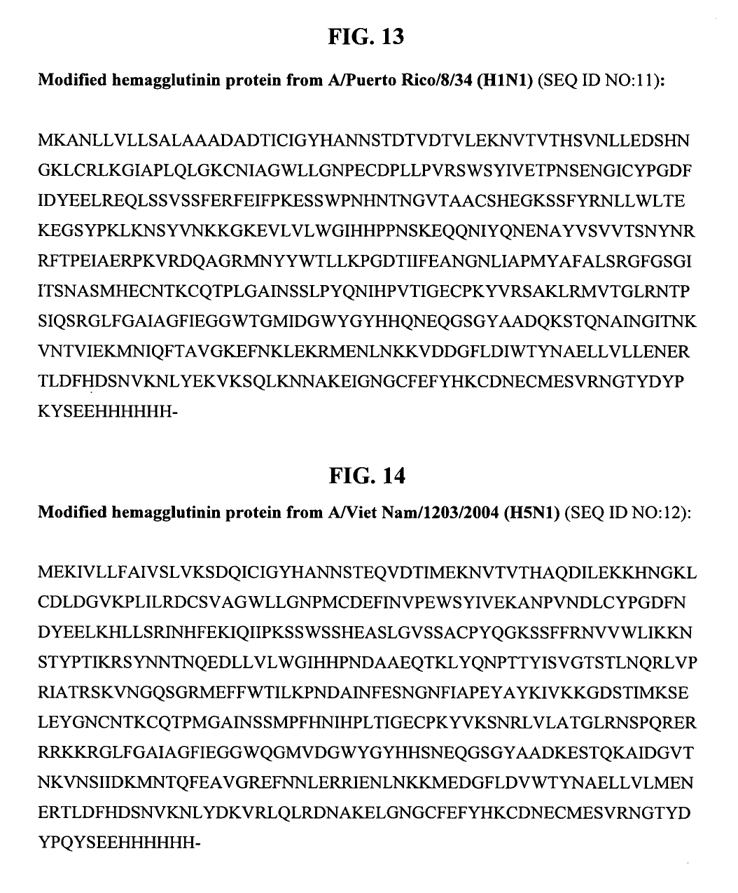

As an illustrative example, Influenza A surface proteins can be used as viral antigens in the invention compositions and methods. The influenza virus infects cells by binding of hemagglutinin molecules to carbohydrate on glycoproteins of host epithelial cells. The virus is engulfed by receptor mediated endocytosis, and a drop in pH within the endocytic vesicle produces a change in structure of the viral hemagglutinin, enabling fusion of the viral membrane with the vesicle membrane. The exposed portion of the hemagglutinin (HA) protein is the ectodomain, which encompasses both the HA1 and HA2 subparts of the protein. Different strains of influenza viruses express HA ectodomain proteins with different amino acid sequences. For example, FIGS. 13 and 14, respectively, show the amino acid sequences of HA ectodomain proteins of A/Puerto Rico/8/34 (from the H1N1 strain) (SEQ ID NO:11) and A/Vietnam/1203/2004 (H₅N1) (SEQ ID NO:12) with modifications to remove the natural signal sequence and add a carboxy terminal His₆ tag for purification according to the invention vaccine assembly methods.

On endocytosis of a virion into endosomes, the viral M2 ion channel is thought to cause acidification of the virion interior. After fusion of the viral membrane with the vesicle membrane, the contents of the virus move to the cytosol. Viral RNA then enters the nucleus of the cell where replication occurs. The replicons return to the cytosol and are translated into the proteins of new virus particles. The influenza virus M2 ion channel is thought to function in the exocytic pathway as well by equilibrating the pH gradient between the acidic lumen of the trans-Golgi network and the neutral cytoplasm. Upon viral budding, only the small ectodomain is exposed on the viral surface. Detachment of the budded virus is aided by the neuraminidase, thus spreading the infection to new cells.

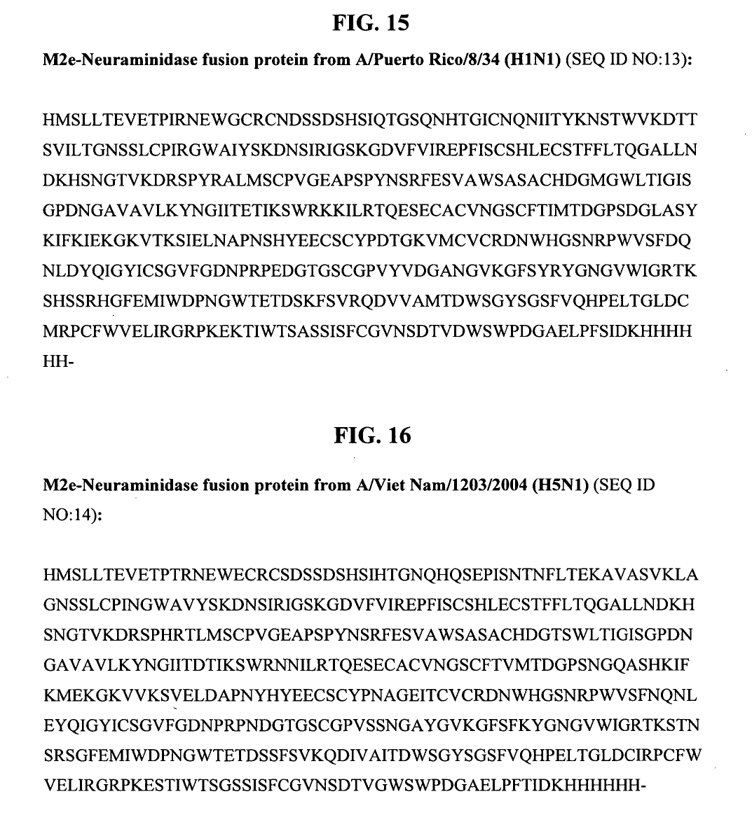

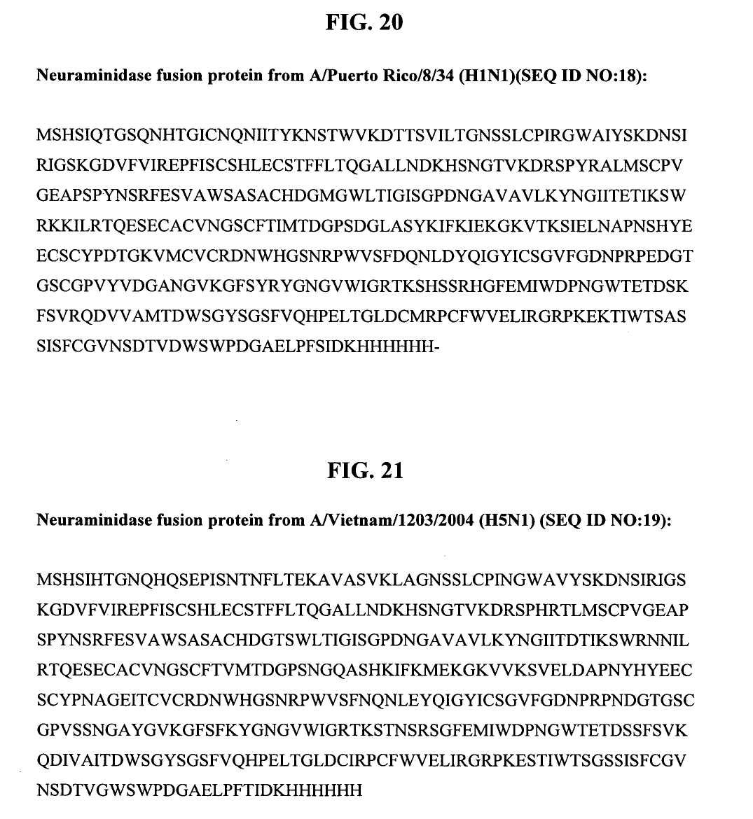

For the invention influenza vaccine, neuraminidase of each of these influenza strains has been fused, via recombinant genetic technology, with the M2 viral membrane protein to form a new antigenic entity. This fusion protein consists of the amino-terminal 24 amino acids of the viral M2 protein (M2e) fused at its carboxy terminus to the ectodomain of the type II membrane protein, neuraminidase (NA). Thus, the NA protein portion lacks its amino terminus, including the membrane-spanning segment thereof. The resultant fusion proteins have been engineered to contain a carboxy-terminal His₆ tag for purification and use in the invention method for assembling a vaccine delivery composition (SEQ ID NO: 13, FIG. 15 and SEQ ID NO:14, FIG. 16). The NA protein ectodomain can also be expressed independently (SEQ ID NO:18, FIG. 20 and SEQ ID NO:19, FIG. 21) and used in a vaccine composition.

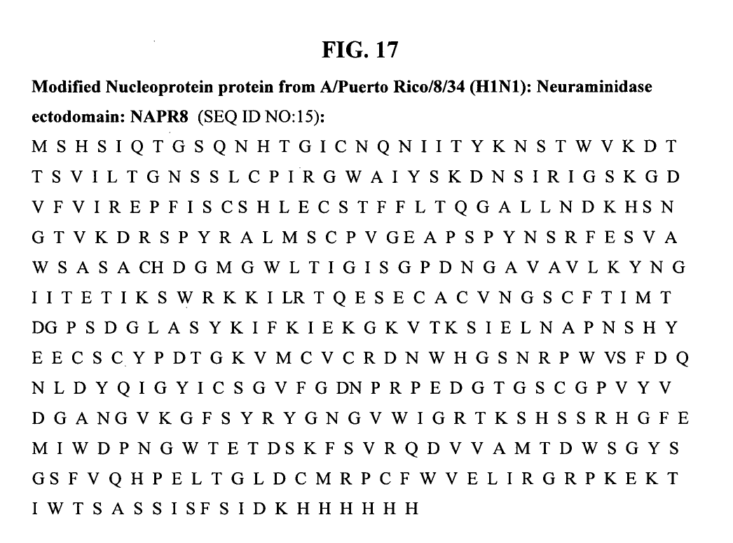

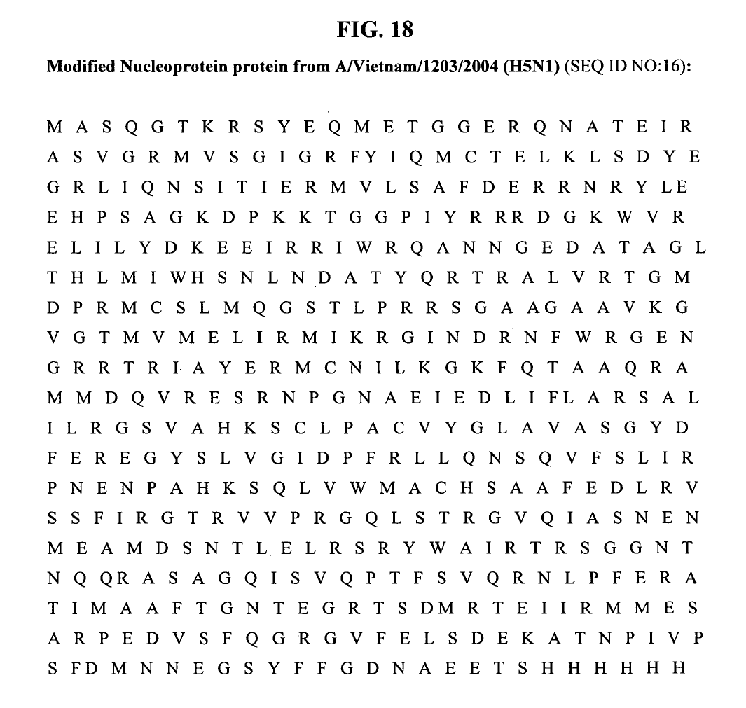

Additional exemplifying influenzan antigens are the nucleoproteins (NP) that are required for encapsidation of the RNA viral genome. These proteins are attractive vaccine components because, like the extracellular portion of M2, the amino acid composition of NP is more highly conserved than the virion surface proteins and function of the NP is also vital to propagate a productive influenza infection. Inclusion of this antigen in an invention composition with one or more of the other influenzan antigens, such as HA, can serve to provide a more comprehensive immune response and thus serve to produce a more potent vaccine. The amino acid sequence of nucleoprotein protein from A/Puerto Rico/8/34 (H1N1) as modified for use in the invention compositions and methods is shown in SEQ ID NO:15, FIG. 17 herein. The similarly modified Nucleoprotein protein from A/Vietnam/1203/2004 (H5N1) is shown in SEQ ID NO:16, FIG. 18 herein.

The compositions and methods described herein will also find use with numerous bacterial antigens, such as those derived from organisms that cause diphtheria, cholera, tuberculosis, tetanus, pertussis, meningitis, Lyme's disease and other pathogenic organisms, including, without limitation, Meningococcus A, B and C, Hemophilus influenza type B (HIB), and Helicobacter pylori. Examples of parasitic antigens include those derived from organisms causing malaria and schistosomiasis.

Furthermore, the methods described herein provide a means for assembly of vaccine compositions for delivering antigens and/or for raising an immune response against a variety of malignant cancers. For example, the compositions prepared by the invention methods can be used to mount both humoral and cell-mediated immune responses to particular antigens specific to the cancer in question, such as an activated oncogene, a fetal antigen, or an activation marker. Such tumor antigens include any of the various MAGEs (melanoma associated antigen E), including MAGE 1, 2, 3, 4, etc. (Boon, T. Scientific American (March 1993):82-89); any of the various tyrosinases; MART 1 (melanoman antigen recognized by T cells), mutant ras; mutant p53; p97 melanoman antigen; CEA (carcinoembryonic antigen), among others. Additional melanoman antigens useful in the preparation of vaccine delivery compositions according to the invention methods and compositions include the following:

| | ANTIGEN | |
|---|---|---|
| DESIGNATION | SEQUENCE | PROTEIN |
| Mart1-27 | AAGIGILTV | MART1 |
| | (SEQ ID NO:11) | |
| Gp100-209* | ITDQVPFSV | Melanocyte lineage- |
| | (SEQ ID NO:12) | specific antigen GP100 |
| Gp100-154 | KTWGQYWQV | Melanocyte lineage- |
| | (SEQ ID NO:13) | specific antigen GP100 |
| Gp100-280 | YLEPGPVTA | Melanocyte lineage- |
| | (SEQ ID NO:14) | specific antigen GP100 |

*GP100 is also called melanoma-associated ME20 antigen.

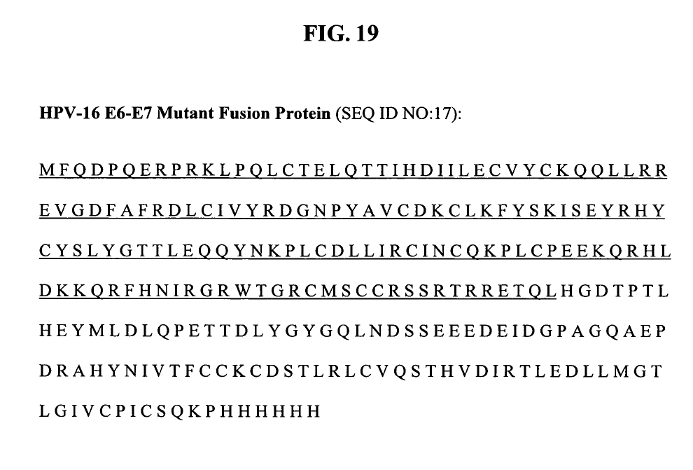

Certain malignancies in humans and animals are associated with viruses that infect T cells and cause those cells to undergo malignant transformation into tumor cells. For example, certain subtypes of HPV are strongly associated with the development of cervical carcinomas, such that nearly every patient with cervical cancer is infected with a papillomavirus. Other subtypes of HPV are associated with genital warts. Given prophylactically, a vaccine that induces a protective immune response against HPV, either humoral or cell-mediated, such that viral infection of cells is blocked, could protect patients from subsequent exposure. A great number of individuals already carry one or more HPV viruses, and transmission rates are high, such that as many as 50% of the sexually active individuals in the United States are postulated to become infected at some point in their lives. For this reason, the development of a therapeutic HPV vaccine is vital. Such a vaccine might be designed so that the intended patient is an individual who has tested positive for the presence of HPV, but has no current symptoms, or it might be designed for the treatment of women who are discovered to have HPV-associated pre-cancerous lesions, or it might be designed for the treatment of women who have early or late stage cervical cancer. Therapeutic vaccines are vaccines given to a patient who is already infected with a pathogen, in some cases a chronic viral pathogen such as Hepatitis C Virus (HCV) or Human Immunodeficiency Virus. In this instance, proteins expressed by the latent or chronic viral infection would be an appropriate vaccine target. In the case of Human Papilloma virus, two proteins, E6 and E7, are expressed in HPV-infected cells and are also expressed in tumor cells arising from such an infection. An invention vaccine composition, therefore, can contain these proteins as well as certain glycolipids, membrane lipids or nucleic acids, coupled to the PEA-NTA. The results of animal studies in which animals were treated with invention vaccine delivery compositions comprising an HPV-16 E6-E7 mutant fusion protein (SEQ ID NO:17, FIG. 19) are presented in the Examples.

It is readily apparent that the subject invention can be used to assemble vaccines against a wide variety of diseases.

The antigens dispersed within the polymers in the invention methods for preparing vaccine delivery compositions can have any suitable length, but may incorporate a peptidic antigen segment of 8 to about 30 amino acids that is recognized by a peptide-restricted T-lymphocyte. Specifically, the antigen segment that is recognized by a corresponding class I peptide-restricted cytotoxic T cell contains 8 to about 12 amino acids, for example 9 to about 11 amino acids and, the antigen segment that is recognized by a corresponding class II peptide-restricted T-helper cell contains 8 to about 30 amino acids, for example about 12 to about 24 amino acids.

While natural T cell mediated immunity works via presentation of peptide epitopes by MHC molecules (on the surface of APCs), MHCs can also present peptide adjunct—in particular glycol-peptides and lipo-peptides, in which the peptide portion is held by the MHC so as to display to the T cell the sugar or lipid moiety. This consideration is particularly relevant in cancer vaccinology because several tumors over-express glyco-derivatized proteins or lipo-derivatized proteins, and the glyco- or lipo-derivatized peptide fragments of these can, in some cases, be powerful T cell epitopes. Moreover, the lipid in such T cell epitopes can be a glyco-lipid.

Unlike the normal peptide-alone presentation, in these cases T cell recognition is dominated by the sugar or lipid group on the peptide, so much so that short synthetic peptides that bind to MHCs with high affinity, but were not derived from the tumor proteins, yet to which the tumor-associated sugar or lipid molecule is covalently attached synthetically, have been successfully used as antigens. This approach to building an artificial T cell epitope directed against a natural tumor cell line has recently been adopted by Franco et al., J. Exp. Med (2004) 199(5):707-716. Therefore, synthetic peptide derivatives and even peptidomimetics can be substituted for the antigen in the invention methods for preparation of vaccine delivery compositions to act as high-affinity MHC-binding ligands that form a platform for the presentation to T cells of peptide branches and non-antigens.

Accordingly, the term “antigen”, as used herein, refers to molecules and portions thereof which are specifically bound by a specific antibody or specific T lymphocyte. Antigens can be proteins, peptides, wholly peptide derivatives (such as branched peptides) and covalent hetero- (such as glyco- and lipo- and glycolipo-) derivatives of peptides. It also is intended to encompass non-peptide molecules that are associated with pathogens or aberrant cells, including, but not limited to, bacterial or viral coat polysaccharides, glycolipids, lipopolysaccharides, oligonucleotides, and phosphate-bearing antigens (phosphoantigens). Fragments of such materials as well as modifications and fusion proteins containing such modified sequences, but which are specifically bound by a specific antibody or specific T lymphocyte are also intended to be encompassed by the term “antigen” as used herein.

The antigens can be synthesized using any technique as is known in the art. The antigens can also include “peptide mimetics.” Peptide analogs are commonly used in the pharmaceutical industry as non-peptide bioactive agents with properties analogous to those of the template peptide. These types of non-peptide compound are termed “peptide mimetics” or “peptidomimetics.” Fauchere, J. (1986) Adv. Bioactive agent Res., 15:29; Veber and Freidinger (1985) TINS p. 392; and Evans et al. (1987) J. Med. Chem., 30:1229; and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, —CH₂—H₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂ NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), and others.

Additionally, substitution of one or more amino acids within a peptide (e.g., with a D-Lysine in place of L-Lysine) may be used to generate more stable peptides and peptides resistant to endogenous proteases. Alternatively, the synthetic antigens, e.g., non-covalently bound to the biodegradable polymer, can also be prepared from D-amino acids, referred to as inverso peptides. When a peptide is assembled in the opposite direction of the native peptide sequence, it is referred to as a retro peptide. In general, peptides prepared from D-amino acids are very stable to enzymatic hydrolysis. Many cases have been reported of preserved biological activities for retro-inverso or partial retro-inverso peptides (U.S. Pat. No. 6,261,569 B1 and references therein; B. Fromme et al., Endocrinology (2003)144:3262-3269.

One or more of the selected antigens is complexed with the biodegradable polymer, with or without adjuvant, for subsequent administration to a subject, as described herein. Once the vaccine delivery composition has been prepared, the composition can be formulated for various delivery routes, including, but not limited to, intravenous, mucosal, intramuscular, or subcutaneous delivery routes. For example, useful polymers in the methods described herein include, but are not limited to, the PEA, PEUR and PEU polymers as described herein. These polymers can be fabricated in a variety of molecular weights, and the appropriate molecular weight for use with a given antigen is readily determined by one of skill in the art. Thus, e.g., a suitable molecular weight will be on the order of about 5,000 to about 300,000 kilodaltons (KD), for example about 5,000 to about 250,000, or about 65,000 to about 200,000, or about 100,000 to about 150,000.

In some embodiments, the persistence, protection, and delivery of the antigen into APCs, by the polymer composition itself may be sufficient to provide immunogenic adjuvant activity. In other embodiments the invention vaccine delivery composition may be prepared to include an adjuvant that can augment immune responses, especially cellular immune responses, to soluble protein antigen, by increasing delivery of antigen, stimulating cytokine production, and/or stimulating antigen presenting cells. Alternatively, the adjuvants can be administered concurrently with the vaccine delivery composition of the invention, e.g., in the same composition or in separate compositions. For example, an adjuvant can be administered prior or subsequent to the vaccine delivery composition of the invention. Alternatively still, the adjuvant can be dispersed in the polymer or an adjuvant/antigen can be non-covalently bonded to the polymer as described herein for simultaneous delivery.

Suitable types of adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides or bacterial cell wall components), such as for example (a) MF59 (International Publication No. WO 90/14837), containing 5% Squalene, 0.5% Tween 80™, and 0.5% Span 85, optionally containing various amounts of MTP-PB, formulated into submicron particles using a microfluidizer such as Model 110Y microfluidizer (Microfluidics, Newton, Mass.), (b) SAF, containing 10% Squalane, 0.4% Tween 80™, 5% pluronic-blocked polymer L121, and thr-MDP, either microfluidized into a submicron emulsion or vortexed to generate a larger particle size emulsion, and (c) Ribi™ adjuvant composition (RAS), (Ribi Immunochem, Hamilton, Mont.) containing 2% Squalene, 0.2% Tween 80™, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2 etc.), macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. W093/13202 and W092/19265); and (7) QS21, a purified form of saponin and 3D-monophosphoryl lipid A (MPL), a nontoxic derivative of lipopolysaccharide (LPS), to enhance cellular and humoral immune responses (Moore, et al., Vaccine. 1999 Jun. 4;17(20-21):2517-27). Other substances such as bacterial, viral or synthetic RNA or DNA compounds (e.g., polyI:C or CpG), carbohydrates or other Toll-Like-Receptor (TLR) ligands that act as immunostimulating adjuvants, may also be used to enhance the effectiveness of the compositions prepared according to the invention methods.

Polymers suitable for use in the practice of the invention bear functionalities that allow facile attachment of the affinity ligand to the polymer. For example, a polymer bearing free amino or carboxyl groups can readily react with a monoclonal antibody or an affinity ligand described herein for use in the invention methods, to conjugate the affinity ligand to the polymer. As will be described herein, the biodegradable polymer and the affinity ligand may contain numerous complementary functional groups that can be used to conjugate the affinity ligand to the biodegradable polymer for the purpose of simultaneously purifying the antigen or and optional adjuvant from a cell lysate other synthetic solution or dispersion while forming the vaccine delivery composition.

The polymer in the invention vaccine delivery composition plays an active role in the endogenous immune processes at the site of implant by holding the antigen and optional adjuvant at the site of injection for a period of time sufficient to allow the individual's immune cells to interact with the antigen and optional adjuvant to affect immune processes, while slowly releasing the particles or polymer molecules containing such agents during biodegradation of the polymer. The fragile antigen and optional adjuvant is protected by the more slowly biodegrading polymer to increase half-life and persistence of the antigen. The co-localization of the antigen and the optional adjuvant can also favorably modulate the host's immune response to the vaccine formulation.

The polymer itself may also have an active role in delivery of the antigen into APCs by stimulating phagocytosis of the polymer-antigen composition. In addition, the polymers disclosed herein, e.g., those having structural formulae (I and III-VIII), upon enzymatic degradation, provide essential amino acids that nurture cells while the other breakdown products can be metabolized using pathways analogous to those used in metabolizing fatty acids and sugars. Uptake of the polymer with antigen/metal ion/affinity ligand complex is safe: studies have shown that the APCs survive, function normally, and can metabolize/clear the degradation products of the invention compositions. These polymers and the vaccine delivery composition produced by the invention methods are, therefore, substantially non-inflammatory to the subject both at the site of injection and systemically, apart from trauma caused by injection itself. Moreover, in the case of active uptake of polymer by APCs, the polymer may act as a delivery adjuvant for the antigen, so there is no essential requirement to formulate an additional adjuvant separately.

Although the invention methods for assembly of delivery compositions are illustrated herein with reference to formation of vaccine delivery compositions with immunogenic and therapeutic utility, the methods described herein can also be used for one-step assembly of compositions for in vivo delivery of a variety of therapeutic biologics so as to substantially retain the native activity and, hence, therapeutic utility of the biologic molecule(s).

The term “therapeutic biologic” is used herein to refer to synthetic or naturally occurring molecules that occur in the mammalian body or affect a bodily process and can be used to a therapeutic end. Specifically included in the meaning of the term are a variety of factors useful in biological processes as well as polymeric macromolecules, such as proteins, polypeptides, as well as all types of DNA and RNA.

It is well known in the art that nucleotides are metal-binding molecules (see, e.g., Wacker E C and Vallee B T, Journal of Biological Chemistry (1959) 234(12):3257-3262). Therefore, in the case of DNA and RNA, the synthetic molecule to be incorporated into the invention delivery composition can be synthesized to contain a nucleotide tag (i.e., modified), rather than an amino-acid containing tag. For, example, in fabrication of the invention compositions for delivery of a strand of RNA or DNA as the therapeutic biologic, the RNA or DNA is conjugated to the polymer active groups via a nucleotide containing tag in the molecule containing the therapeutic biologic at either the 3′ or the 5′ end. These procedures, examples of which are illustrated schematically below, can also be used to synthesize His-tagged biologics, in which the non-tag portion is not a peptide or protein, but is a polynucleotide (RNA or DNA), a polysaccharide, a lipid or a small molecule hapten.

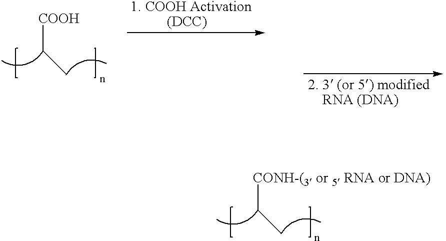

It is well known in the art that nucleosides and nucleotides bind transition metals, and that the base moiety of purines in particular binds the metal cation in a manner analagous to the binding by Histidine (see, e.g. De Meester P, et al., Biochem. J., (1974) 134, 791-792; Collins A D, et al., Biochim Biophys Acta, 402(1):1-6, 1975; Goodgame D M L, et al., Nucleic Acids Res., 2(8):1375-1379, 1975; Gao Y-G, et al., Nucleic Acids Res., 21(17):4093-4101, 1993). Thus, polynucleotide adjuvant molecules, such as CpG or polyI:C can be incorporated directly into the vaccine particle, with or without accompanying antigen, by binding to the transition metal.

The biodegradable polymers useful in forming the invention biocompatible delivery compositions include those comprising at least one amino acid conjugated to at least one non-amino acid moiety per monomer. The term “non-amino acid moiety” as used herein includes various chemical moieties, but specifically excludes amino acid derivatives and peptidomimetics as described herein. In addition, the polymers containing at least one amino acid are not contemplated to include polyamino acid segments, including naturally occurring polypeptides, unless specifically described as such. In one embodiment, the non-amino acid is placed between two adjacent amino acids in the monomer. In another embodiment, the non-amino acid moiety is hydrophobic. The polymer may also be a block co-polymer.

Preferred polymers for use in the invention compositions and methods are polyester amides (PEAs) polyester urethanes (PEURs) and polyester ureas (PEUs) that have built-in functional groups on the polymer backbone, and these built-in functional groups can react with other chemicals and lead to the incorporation of additional functional groups to expand the functionality of the polymers further. Therefore, such polymers used in the invention methods are also ready for reaction with other chemicals having a hydrophilic structure to increase water solubility and with antigens, adjuvants, and other agents, without the necessity of prior modification.

In addition, the PEA, PEUR and PEU polymers used in preparation of the invention delivery compositions display no hydrolytic degradation when tested in a saline (PBS) medium, but in an enzymatic solution, such as chymotrypsin, a uniform erosive behavior has been observed, resulting in controlled delivery of the antigen.

Accordingly, in one embodiment the polymer used in the invention methods comprises at least one or a blend of the following: a PEA having a chemical formula described by structural formula (I), 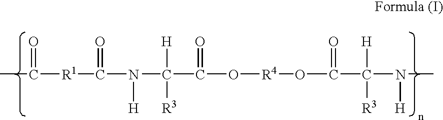
wherein n ranges from about 5 to about 150; R¹ is independently selected from residues of α,ω-bis(4-carboxyphenoxy)-(C₁-C₈) alkane, 3,3′-(alkanedioyldioxy)dicinnamic acid or 4,4′-(alkanedioyidioxy)dicinnamic acid, (C₂-C₂₀) alkylene, or (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof, (C₂-C₂₀) alkylene, and (C₂-C₂₀) alkenylene;

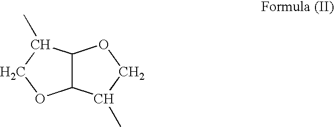

or a PEA having a chemical formula described by structural formula III: 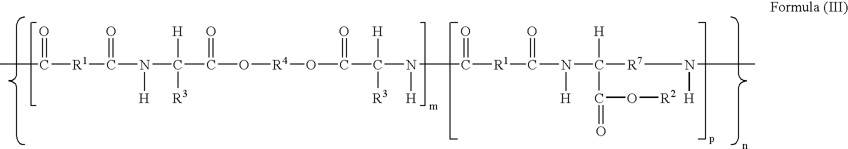
wherein n ranges from about 5 to about 150, m ranges about 0.1 to 0.9:p ranges from about 0.9 to 0.1; wherein R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol or bicyclic-fragment of 1,4:3,6-dianhydrohexitols of structural formula(II), and combinations thereof; and R⁷ is independently (C₁-C₂₀) alkyl or (C₂-C₂₀) alkenyl, for example, (C₃-C₆) alkyl or (C₃-C₆) alkenyl;

or a PEUR having a chemical formula described by structural formula (IV), 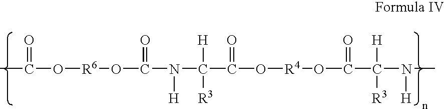
wherein n ranges from about 5 to about. 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀) alkylene, (C₂-C₂₀) alkenylene or alkyloxy, bicyclic-fragments of 1,4:3,6-dianhydrohexitols of general formula (II), and combinations thereof;

or a PEUR having a chemical structure described by general structural formula (V) 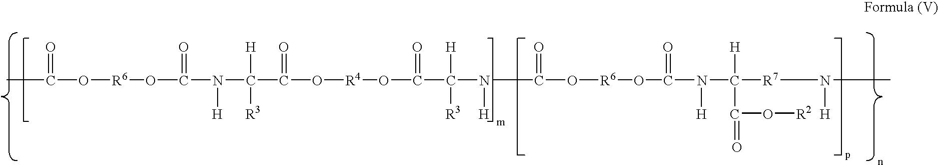
wherein n ranges from about 5 to about 150, m ranges about 0.1 to about 0.9:p ranges from about 0.9 to about 0.1; R² is independently selected from hydrogen, (C₆-C₁₀) aryl (C₁-C₂₀) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II) and combinations thereof; R⁶ is independently selected from (C₂-C₂₀) alkylene, (C₂-C₂₀) alkenylene or alkyloxy, bicyclic-fragments of 1,4:3,6-dianhydrohexitols of general formula (II), a residue of a saturated or unsaturated therapeutic diol, and combinations thereof; and R⁷ is independently (C₁-C₂₀) alkyl or (C₂-C₂₀) alkenyl;

or a PEU having a chemical formula described by general structural formula (VI): 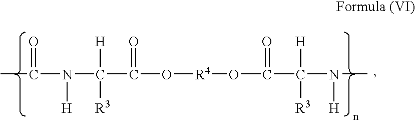
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II);

or a PEU having a chemical formula described by structural formula (VII) 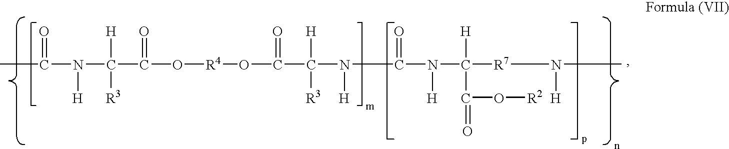
wherein m is about 0.1 to about 1.0; p is about 0.9 to about 0.1; n is about 10 to about 150; each R² is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; a bicyclic-fragment of a 1,4:3,6-dianhydrohexitol of structural formula (II), and combinations thereof; and R⁷ is independently (C₁-C₂₀) alkyl or (C₂-C₂₀) alkenyl, for example, (C₃-C₆) alkyl or (C₃-C₆) alkenyl.

For example, in one alternative in the PEA polymer used in the invention method for assembly of a polymer-based delivery composition, at least one R¹ is a residue of α,ω-bis(4-carboxyphenoxy) (C₁-C₈) alkane, 3,3′-(alkanedioyldioxy)dicinnamic acid, or 4,4′-(alkanedioyldioxy)dicinnamic acid and R⁴ is a bicyclic-fragment of a 1,4:3,6-dianhydrohexitol of general formula(II). In another alternative, R¹ in the PEA polymer is either a residue of α,ω-bis(4-carboxyphenoxy) (C₁-C₈) alkane, 3,3′-(alkanedioyldioxy)dicinnamic acid, or 4,4′-(alkanedioyldioxy)dicinnamic acid. In yet another alternative, in the PEA polymer R¹ is a residue α,ω-bis (4-carboxyphenoxy) (C₁-C₈) alkane, such as 1,3-bis(4-carboxyphenoxy)propane (CPP), 3,3′-(alkanedioyldioxy)dicinnamic acid or 4,4′-(adipoyldioxy)dicinnamic acid and R⁴ is a bicyclic-fragment of a 1,4:3,6-dianhydrohexitol of general formula (II), such as DAS.

Preferably R⁷ is —(CH₂)₄—.

Suitable protecting groups for use in practice of the invention include t-butyl and others as are known in the art. Suitable bicyclic-fragments of 1,4:3,6-dianhydrohexitols can be derived from sugar alcohols, such as D-glucitol, D-mannitol, and L-iditol. For example, 1,4:3,6-dianhydrosorbitol (isosorbide, DAS) is particularly suited for use as a bicyclic-fragment of 1,4:3,6-dianhydrohexitol.

PEU polymers, as described herein, can be fabricated as high molecular weight polymers useful for making the invention delivery compositions for delivery to humans and other mammals. The PEUs used in the invention methods incorporate hydrolytically cleavable ester groups and non-toxic, naturally occurring monomers that contain α-amino acids in the polymer chains. The ultimate biodegradation products of PEUs will be α-amino acids (whether biological or not), diols, and CO₂. In contrast to the PEAs and PEURs, PEUs are crystalline or semi-crystalline and possess advantageous mechanical, chemical and biodegradation properties that allow formulation of completely synthetic, and hence easy to produce, mesoscopic range polymer particles, for example nanoparticles.

For example, the PEU polymers used in the invention method for preparation of delivery compositions have high mechanical strength, and surface erosion of the PEU polymers can be catalyzed by enzymes present in physiological conditions, such as hydrolases.

In one alternative in the PEU polymer, at least one R⁴ is a bicyclic fragment of a 1,4:3,6-dianhydrohexitol, such as 1,4:3,6-dianhydrosorbitol (DAS).

In one alternative, the R³s in at least one n monomer of the polymers of Formulas (I and III-VII are CH₂Ph and the α-amino acid used in synthesis is L-phenylalanine. In alternatives wherein the R³s within a monomer are —CH₂—CH(CH₃)₂, the polymer contains the α-amino acid, leucine. By varying the R³s, other α-amino acids can also be used, e.g., glycine (when the R³s are —H), alanine (when the R³s are —CH₃), valine (when the R³s are —CH(CH₃)₂), isoleucine (when the R³s are —CH(CH₃)—CH₂—CH₃), phenylalanine (when the R³s are —CH₂—C₆H₅); lysine (when the R³s are —(CH₂)₄—NH₂); or methionine (when the R³s are —(CH₂)₂SCH₃).

In yet a further embodiment wherein the polymer is a PEA, PEUR or PEU of formula I or III-VII, at least one of the R³s further can be —(CH₂)₃— wherein the R³s cyclize to form the chemical structure described by structural formula (XIII):

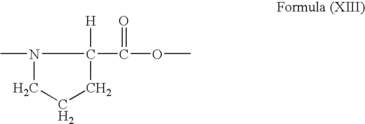

When the R³s are —(CH₂)₃—, an α-imino acid analogous to pyrrolidine-2-carboxylic acid (proline) is used.

The PEAs, PEURs and PEUs are biodegradable polymers that biodegrade substantially by enzymatic action so as to release the dispersed antigen and optional adjuvant over time. Due to structural properties of these polymers, when used in the invention methods, the vaccine delivery compositions so formed provide for stable loading of the antigens and optional adjuvants while preserving the three dimensional structure thereof and, hence, the bioactivity.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, proline, or a mixture thereof.

In the PEA, PEUR and PEU polymers useful in practicing the invention, multiple different α-amino acids can be employed in a single polymer molecule. These polymers may comprise at least two different amino acids per repeat unit and a single polymer molecule may contain multiple different α-amino acids in the polymer molecule, depending upon the size of the molecule. In one alternative, at least one of the α-amino acids used in fabrication of the invention polymers is a biological α-amino acid.

The term “aryl” is used with reference to structural formulae herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain.

As used herein, a “therapeutic diol” means any diol molecule, whether synthetically produced, or naturally occurring (e.g., endogenously) that affects a biological process in a mammalian individual, such as a human, in a therapeutic or palliative manner when administered.

As used herein, the term “residue of a therapeutic diol” means a portion of a therapeutic diol, as described herein, which portion excludes the two hydroxyl groups of the diol. The corresponding therapeutic diol containing the “residue” thereof is used in synthesis of the polymer compositions. The residue of the therapeutic diol is reconstituted in vivo (or under similar conditions of pH, aqueous media, and the like) to the corresponding diol upon release from the backbone of the polymer by biodegradation in a controlled manner that depends upon the properties of the PEA, PEUR or PEU polymer selected to fabricate the composition, which properties are as known in the art and as described herein.

Due to the versatility of the PEA, PEUR and PEU polymers used in the invention compositions, the amount of the therapeutic diol incorporated in the polymer backbone can be controlled by varying the proportions of the building blocks of the polymer. For example, depending on the composition of the PEA, loading of up to 40% w/w of 17β-estradiol can be achieved. Two different regular, linear PEAs with various loading ratios of 17β-estradiol are illustrated in Scheme 1 below:

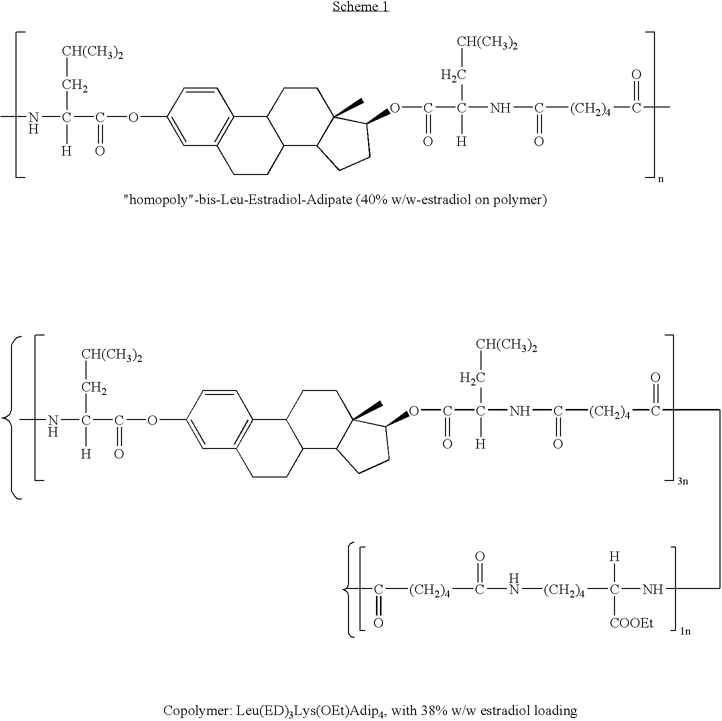

Similarly, the loading of the therapeutic diol into PEUR and PEU polymer can be varied by varying the amount of two or more building blocks of the polymer.

In addition, synthetic steroid based diols based on testosterone or cholesterol, such as 4-androstene-3,17 diol (4-Androstenediol), 5-androstene-3,17 diol (5-Androstenediol), 19-nor5-androstene-3,17 diol (19-Norandrostenediol) are suitable for incorporation into the backbone of PEA and PEUR polymers according to this invention. Moreover, therapeutic diol compounds suitable for use in preparation of the invention polymer particle delivery compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycin A; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

Methods for making polymers such as those of structural formulas (I) and (III-VII), which contain an α-amino acid in the general formula, are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine, for example, by condensing the α-amino acid containing pendant R³ with a diol HO—R⁴—OH. As a result, di-ester monomers with reactive α,ω-amino groups are formed. Then, the bis-α,ω-diamine is entered into a polycondensation reaction with a di-acid such as sebacic acid, or its bis-activated esters, or bis-acyl chlorides, to obtain the final polymer having both ester and amide bonds (PEA). Alternatively, for PEUR, instead of the di-acid, a di-carbonate derivative, formula (VIII), is used, where R⁶ is defined above and R⁸ is independently (C₆-C₁₀)aryl, optionally substituted with one or more of nitro, cyano, halo, trifluoromethyl or trifluoromethoxy.

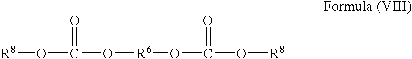

More particularly, synthesis of the unsaturated poly(ester-amide)s (UPEAs) useful as biodegradable polymers of the structural formula (I) as disclosed above will be described, wherein 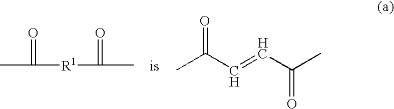
and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis (α-amino acid) diesters, comprising at least 1 double bond in R⁴, and di-p-nitrophenyl esters of saturated dicarboxylic acid or (2) di-p-toluene sulfonic acid salt of bis (α-amino acid) diesters, comprising no double bonds in R⁴, and di-nitrophenyl ester of unsaturated dicarboxylic acid or (3) di-p-toluene sulfonic acid salt of bis(α-amino acid) diesters, comprising at least one double bond in R⁴, and di-nitrophenyl esters of unsaturated dicarboxylic acids.

Salts of p-toluene sulfonic acid are known for use in synthesizing polymers containing amino acid residues. The aryl sulfonic acid salts are used instead of the free base because the aryl sulfonic salts of bis (α-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

The di-p-nitrophenyl esters of unsaturated dicarboxylic acid can be synthesized from p-nitrophenol and unsaturated dicarboxylic acid chloride, e.g., by dissolving triethylamine and p-nitrophenol in acetone and adding unsaturated dicarboxylic acid chloride drop wise with stirring at −78° C. and pouring into water to precipitate product. Suitable acid chlorides useful for this purpose include fumaric, maleic, mesaconic, citraconic, glutaconic, itaconic, ethenyl-butane dioic and 2-propenyl-butanedioic acid chlorides.

The di-aryl sulfonic acid salts of bis(α-amino acid) diesters can be prepared by admixing α-amino acid, p-aryl sulfonic acid (e.g. p-toluene sulfonic acid monohydrate), and saturated or unsaturated diol in toluene, heating to reflux temperature, until water evolution is minimal, then cooling. The unsaturated diols useful for this purpose include, for example, 2-butene-1,3-diol and 1,18-octadec-9-en-diol.

Saturated di-p-nitrophenyl esters of dicarboxylic acids and saturated di-p-toluene sulfonic acid salts of bis(α-amino acid) di-esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B1, except that R⁴ of (III) of U.S. Pat. No. 6,503,538 and/or R¹ of (V) of U.S. Pat. No. 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis(L-phenylalanine)-2-butenediol-1,4-diester for (III) in Example 1 of U.S. Pat. No. 6,503,538 or by substituting di-p-nitrophenyl fumarate for (V) in Example 1 of U.S. Pat. No. 6,503,538 or by substituting p-toluene sulfonic acid salt of bis(L-phenylalanine)-2-butenediol-1,3-diester for III in Example 1 of U.S. Pat. No. 6,503,538 and also substituting de-p-nitrophenyl fumarate for (V) in Example 1 of U.S. Pat. No. 6,503,538.

In unsaturated compounds having either structural formula (I) or (III), the following hold: Aminoxyl radical e.g., 4-amino TEMPO, can be attached using carbonyldiimidazol, or suitable carbodiimide, as a condensing agent. Antigens, adjuvants and antigen/adjuvant conjugates or fusion proteins, as described herein, can be attached via the double bond functionality. Hydrophilicity can be imparted by bonding to poly(ethylene glycol) diacrylate.

In yet another aspect, polymers contemplated for use in forming the invention methods for assembly of delivery compositions include those set forth in U.S. Pat. Nos. 5,516,881; 6,476,204; 6,503,538; and in U.S. application Ser. Nos. 10/096,435; 10/101,408; 10/143,572; and 10/194,965; the entire contents of each of which is incorporated herein by reference.

The biodegradable PEA, PEUR and PEU polymers and copolymers may contain up to two amino acids per monomer, multiple amino acids per polymer molecule, and preferably have weight average molecular weights ranging from 10,000 to 125,000; these polymers and copolymers typically have intrinsic viscosities at 25° C., determined by standard viscosimetric methods, ranging from 0.3 to 4.0, for example, ranging from 0.5 to 3.5.

Polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (IX) as follows:

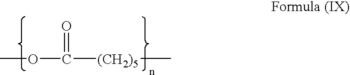

Poly(glycolides) contemplated for use have an exemplary structural formula (X) as follows:

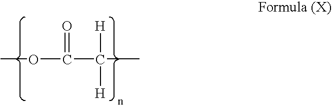

Poly(lactides) contemplated for use have an exemplary structural formula (XI) as follows:

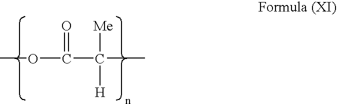

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XII).

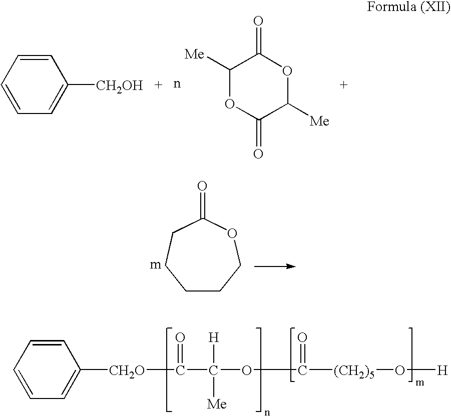

The hydroxy terminated polymer chains can then be capped with maleic anhydride to form polymer chains having structural formula (XIII):

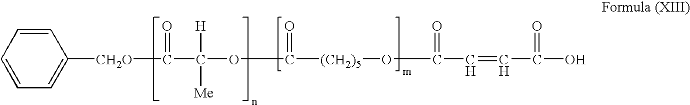

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated compounds having structural formula (VII) for PEU the following hold: An amino substituted aminoxyl (N-oxide) radical bearing group e.g., 4-amino TEMPO, can be attached using carbonyldiimidazole, or suitable carbodiimide, as a condensing agent. Additional bioactive agents, and the like, as described herein, optionally can be attached via the double bond.

For example, the invention high molecular weight semi-crystalline PEUs having structural formula (VI) can be prepared inter-facially by using phosgene as a bis-electrophilic monomer in a chloroform/water system, as shown in the reaction scheme (2) below:

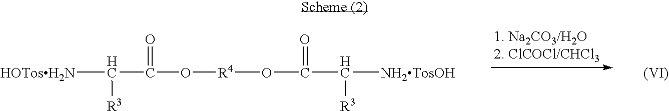

Synthesis of copoly(ester ureas) (PEUs) containing L-Lysine esters and having structural formula (VII) can be carried out by a similar scheme (3):

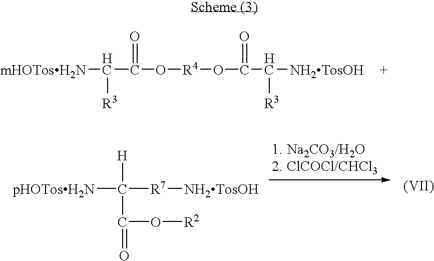

A 20% solution of phosgene (ClCOCl) (highly toxic) in toluene, for example (commercially available (Fluka Chemie, GMBH, Buchs, Switzerland), can be substituted either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

General Procedure for Synthesis of PEUs

It is necessary to use cooled solutions of monomers to obtain PEUs of high molecular weight. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then the chloroform layer can be separated, dried over anhydrous Na₂SO₄, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing the chloroform to evaporate to dryness. No further purification of obtained PEUs is needed. The yield and characteristics of exemplary PEUs obtained by this procedure are summarized in Table 1 herein.

General Procedure for Preparation of Porous PEUs.

Methods for making the PEU polymers containing α-amino acids in the general formula will now be described. For example, for the embodiment of the polymer of formula (I) or (II), the α-amino acid can be converted into a bis-(α-amino acid)-α,ω-diol-diester monomer, for example, by condensing the α-amino acid with a diol HO—R¹—OH. As a result, ester bonds are formed. Then, acid chloride of carbonic acid (phosgene, diphosgene, triphosgene) is entered into a polycondensation reaction with a di-p-toluenesulfonic acid salt of a bis-(α-amino acid)-alkylene diester to obtain the final polymer having both ester and urea bonds.

The unsaturated PEUs can be prepared by interfacial solution condensation of di-p-toluenesulfonate salts of bis-(α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong material, L-Leu based PEUs, such as 1-L-Leu-4 and 1-L-Leu-6, can be fabricated using the general procedure described below. Such procedure is less successful in formation of a porous, strong material when applied to L-Phe based PEUs.

The reaction solution or emulsion (about 100 mL) of PEU in chloroform, as obtained just after interfacial polycondensation, is added dropwise with stirring to 1,000 mL of about 80° C.-85° C. water in a glass beaker, preferably a beaker made hydrophobic with dimethyldichlorsilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 1.

TABLE 1
Properties of PEU Polymers of Formula (VI) and (VII)

| | Yield | η_{red} ^{a)} | | | M_{w}/ | Tg ^{c)} | Tₘ ^{c)} |
|---|---|---|---|---|---|---|---|
| PEU* | [%] | [dL/g] | M_{w} ^{b)} | Mₙ ^{b)} | Mₙ ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L-Leu- | 84 | 0.31 | 64400 | 43000 | 1.47 | 34 | 114 |
| 6]_{0.75}-[1-L- | | | | | | | |
| Lys(OBn)]_{0.25} | | | | | | | |
| 1-L-Leu-DAS | 57 | 0.28 | 55700 ^{d)} | 27700 ^{d)} | 2.1 ^{d)} | 56 | 165 |

*PEUs of general formula (VI), where,
1-L-Leu-4: R⁴ = (CH₂)₄, R³ = i-C₄H₉
1-L-Leu-6: R⁴ = (CH₂)₆, R³ = i-C₄H₉
1-L-Phe-6: R⁴ = (CH₂)₆, R³ = —CH₂—C₆H₅.
1-L-Leu-DAS: R⁴ = 1,4:3,6-dianhydrosorbitol, R³ = i-C₄H
Reduced viscosities were measured in DMF at 25° C. and a concentration 0.5 g/dL
^{b)} GPC Measurements were carried out in DMF, (PMMA)
^{c)} Tg taken from second heating curve from DSC Measurements (heating rate 10° C./min).
^{d)} GPC Measurements were carried out in DMAc, (PS)

Tensile strength of illustrative synthesized PEUs was measured and results are summarized in Table 2. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties: 1. A glass transition temperature in the range from about 30 C.° to about 90 C.°, for example, in the range from about 35 C.° to about 70 C.°; 2. A film of the polymer with average thickness of about 1.6 cm will have tensile stress at yield of about 20 Mpa to about 150 Mpa, for example, about 25 Mpa to about 60 Mpa; 3. A film of the polymer with average thickness of about 1.6 cm will have a percent elongation of about 10% to about 200%, for example about 50% to about 150%; and 4. A film of the polymer with average thickness of about 1.6 cm will have a Young's modulus in the range from about 500 MPa to about 2000 MPa. Table 2 below summarizes the properties of exemplary PEUs of this type.

TABLE 2
Mechanical Properties of PEUs

| | | Tensile | Percent | Young's |
|---|---|---|---|---|
| | Tg^{a)} | Stress at | Elongation | Modulus |
| Polymer designation | (° C.) | Yield (MPa) | (%) | (MPa) |
| 1-L-Leu-6 | 64 | 21 | 114 | 622 |
| [1-L-Leu- | 34 | 25 | 159 | 915 |
| 6]_{0.75}-[1-L- | | | | |
| Lys(OBn)]_{0.25} | | | | |

The various components of the invention delivery composition can be present in a wide range of ratios. For example, the polymer repeating unit:antigen or repeating unit:therapeutic biologic are typically used in a ratio of 1:50 to 50:1, for example 1:10 to 10:1, about 1:3 to 3:1, or about 1:1. However, other ratios may be more appropriate for specific purposes, such as when a particular antigen is both difficult to incorporate into a particular polymer and has a low immunogenicity, in which case a higher relative amount of the antigen is required.

The polymers used in the invention delivery compositions, such as PEA, PEUR and PEU polymers, biodegrade by enzymatic action at the surface. Therefore, the polymers, for example particles thereof, administer the antigen to the subject at a controlled release rate, which is specific and constant over a prolonged period. Additionally, since PEA, PEUR and PEU polymers break down in vivo via hydrolytic enzymes without production of adverse side-products, the invention delivery compositions are substantially non-inflammatory. As used herein, “biodegradable” as used to describe a polymer in the invention delivery compositions means the polymer is capable of being broken down into innocuous products in the normal functioning of the body. In one embodiment, the entire delivery composition is biodegradable. The preferred biodegradable polymers have hydrolyzable ester linkages that provide the biodegradability, and are typically chain terminated predominantly with amino groups.

As used herein “dispersed” means a molecule, such as an antigen or adjuvant, as disclosed herein is dispersed, mixed, dissolved, homogenized, and covalently or non-covalently bound (“dispersed” or loaded) in the polymer, which may or may not be formed into particles. For example, in the invention method for assembly of vaccine a delivery composition, at least one antigen or adjuvant, or both, is non-covalently bound to the polymer via a complex of an affinity ligand that binds specifically to the protein or antigen, for example via a metal affinity complex comprising an affinity ligand, and a transition metal ion. If more than one antigen is desired, multiple antigens or antigens plus adjuvants may be dispersed in individual polymers and then mixed as needed to form the final vaccine delivery composition, or the antigens with or without adjuvants may be mixed together and then dispersed into a single polymer to form the final vaccine delivery composition.

Preparation of Recombinant Protein or Peptide Antigen

Techniques for recombinant production of heterologous polypeptides, including peptide antigens, in organisms, such as bacterial and eukaryotic cell expression systems, are well known in the art and do not bear extensive description in this application. For example, the preparation of the antigens and fusion proteins used in the practice of this invention can be carried out using standard recombinant DNA methods. Preferably, a nucleotide sequence coding for the desired affinity peptide is first synthesized and then is linked to a nucleotide sequence coding for the His tag. A similar method can be used for production of synthetic biologics to be used in the invention methods.

The thus-obtained hybrid gene can be incorporated into expression vectors such as plasmid pDS8/RBSII, SphI; pDS5/RBSII, 3A+5A; pDS78/RBSII; pDS56/RBSII or other commercial or generally accessible plasmids, using standard methods. Most of the requisite methodology can be found in Maniatis et al., “Molecular Cloning”, Cold Spring Harbor Laboratory, 2001, which is hereby incorporated by reference to illustrate the state of the art.

Methods for the expression of the fusion proteins of this invention are also described by Maniatis et al., supra. They embrace the following procedures: (a) Transformation of a suitable host organism, for example E. coli or insect cell line Sf9, with an expression vector in which the hybrid gene is operatively linked to an expression control sequence; (b) Cultivation of the transformed host organism under suitable growth conditions; and (c) Extraction and isolation of the desired fusion protein from the host organism. Host organisms that can be used include but are not limited to insect cell lines, such as Sf9, and Sf21, gram-negative and gram-positive bacteria, such as E. coli and B. subtilis strains, such as E. coli strain M15. Other E. coli strains that can be used include, e.g., E. coli 294 (ATCC No. 3144), E. coli RR1 (ATCC No. 31343) and E. coli W3110 (ATCC No. 27325). Insect cells transformed with baculovirus vectors are presently preferred to insure proper folding of a protein or polypeptide antigen.

Three Methods to Selectively Capture Antigenic Proteins and Antigens from a Recombinant T Cell Lysate.

For production of large quantities of protein antigens and peptides by recombinant gene technologies, coding regions for the proteins are integrated into artificial genes, which are replicated and expressed in bacteria, usually E. coli, or in a virus, such as baculovirus, which replicates in host insect T cells. Whichever method is used, the over-expressed antigens or therapeutic biologic must then be selectively removed from the cell lysate or culture supernatant for subsequent incorporation into a delivery composition.

Three methods are described here for the selective capture of target molecules from cell lysate according to the invention methods. PEA and PEUR polymers of structural formulas III and IV, respectively, have been used to both capture the target molecules containing antigens and, simultaneously, to form the core of the vaccine delivery composition. In this embodiment, the polymer is mixed directly with fresh lysate, resulting in formation of an antigen-polymer complex. Because there is a protein-capture point on every repeat unit of these PEA and PEUR polymers, the antigen-polymer complex molecules are of sufficiently high molecular mass that they can be removed from the remaining lysate by size-filtration.

Oligomerization In this embodiment, the invention vaccine assembly method may be used to capture antigenic proteins that naturally form oligomers. Examples are the functional trimer of hemaglutinin (HA) and the tetramer of neuraminidase (NA) from influenza A virus.

Previously prepared target antigen protomer is conjugated to repeat units of the polymer. The protomer-polymer complex is mixed with lysate under batch conditions that promote oligomerization of the antigenic proteins. The resultant oligomer-polymer complex is removed from the remaining filtrate by size-filtration. A more complete description of preparation of the invention vaccine delivery compositions by the oligomerization technique is contained in U.S. application Ser. No. 11/345,021, filed Jan. 31, 2006.

Antibody (Ab) recognition This method may be used to capture protein and polypeptide antigens against which humanized monoclonal antibody molecules or active fragments thereof (MAbs or FAbs) have been prepared, for example, as described herein.

Previously prepared MAb or FAb molecules against target antigen are conjugated to repeat units of the polymer, either directly using amide bond or cysteine-maleimide bond formation, or indirectly by an incorporated His tag and metal affinity ligand as described herein, or with polymer-conjugated Ab-binding protein domains, such as those from protein A or protein G, which are well known in the art. In this embodiment, the Ab-polymer complex is mixed with lysate under batch conditions that promote antibody binding. The resulting antigen-Ab-polymer complex is removed from the remaining filtrate by size-filtration.

Polymer-Affinity Ligand Linkage

Metal affinity complex formation In this embodiment, repeat units of the polymer are pre-functionalized with suitable metal affinity ligands, such as (A) an imidazole derivative, or (B) an NTA derivative, such as nitrilotriacetic acid (NTA) or iminodiacetic acid (IDA). The affinity ligands are directly conjugated to the biodegradable polymers via a wide variety of suitable functional groups. For example, when the biodegradable polymer is a polyester, the carboxyl group chain end can be used to react with a complimentary moiety on the affinity ligand (e.g., the one or more free amino groups, on the metal affinity ligand NTA or IDA). A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, the affinity ligand can be linked to any of the polymers of structures (I) or (III-VII) through a free amide, ester, ether, amino, ketone, thioether, sulfinyl, sulfonyl, disulfide linkage. Such a linkage can be formed from suitably functionalized starting materials using synthetic procedures that are known in the art. For example, in one embodiment the polymer can be linked to the metal affinity ligand via an end or pendent carboxyl group (e.g., COOH) of the polymer. Specifically, the metal affinity ligand used in the invention methods can react with a polymer with an amino functional group or a hydroxyl functional group of the polymer, such as those described by structural formulas III, V and VII, while leaving free binding sites for forming a coordination complex with a transition metal ion and metal binding amino acids of molecule comprising an antigen to provide a biodegradable polymer having the antigen non-covalently attached to the polymer via a metal affinity complex. In another embodiment, the carboxyl group of the polymer can be transformed into an acyl halide, acyl anhydride/“mixed” anhydride, or active ester. In other embodiments, the free —NH₂ ends of the polymer molecule can be acylated to assure that the affinity ligand will attach only via a carboxyl group of the polymer and not to the free ends of the polymer. For example, the invention vaccine delivery composition described herein can be prepared from PEA, PEUR, or PEU where the N-terminal free amino groups are acylated, e.g., with anhydride RCOOCOR, where the R=(C₁-C₂₄) alkyl, to assure that the antigenic protein or antigen will attach only via an affinity complex formed at a carboxyl group of the polymer and not to the free ends of the polymer.

For example, in one embodiment, side-chain protected lysine (e.g. ε-N-Boc, OBn-Lys) is conjugated via an amide bond to the activated carboxylate on the repeat unit of the PEA, PEUR or PEU polymer of structural formulas III, IV or VII. Following de-protection, the free ε-amino groups of these lysine residues are modified by reaction with a metal affinity ligand, such as 2-imidazolecarboxaldehyde.

A transition metal (TM) selected from Fe²⁺, Cu²⁺, or Ni²⁺ is then bound to the metal affinity ligand, e.g., 2-imidazolecarboxaldehyde. The resultant TM-derivatized polymer is bio-functionalized via the bound TM(II) with a protein bearing antigen, such as one that contains one or more metal-binding amino acid residues, such as Trp or a histidine extension, e.g., a His₆ tag.

The strength of the metal affinity complexes formed varies according to the number and distribution of metal-binding amino acids in the antigen or molecule containing the antigen and the metal ions used. The metal ions used in practice of the invention are nickel (Ni²⁺) copper (Cu²⁺) zinc (Zn²⁺) and cobalt (Co²⁺). In general, the strength of binding of the antigen or fusion protein incorporating the antigen to the metal ion decreases in the following order: Cu²⁺>Ni²⁺>Co²⁺>Zn²⁺.

In this embodiment, the high efficiency of the invention methods for assembly of a delivery composition is based on interaction of a metal affinity ligand, which is conjugated to the polymer, the metal transition ion selected, and the metal-binding amino acids in the target molecule, especially tryptophan (Trp) and histidine (His). The metal affinity ligands suitable for use in the invention methods for assembling a delivery composition include nitrilotriacetic acid (NTA) and iminodiacetic acid (IDA). NTA is a tetradentate metal affinity ligand known to bind to a variety of transition metals with stability constants of 10⁹ to 10¹⁴. The stability constant remains high due to the presence of multiple free metal coordination sites therein after the NTA is conjugated to available functional groups in the polymer. For example, when iminodiacetic acid (IDA) is used as the metal affinity ligand, a bidentate chelating moiety, to which a metal ion can be coordinated, remains free after binding of IDA to the polymer. Various metal ions can be coordinated via these bound metal affinity ligands so that free coordination sites on the metal ions in turn are free to bind to metal binding amino acids in the target molecule. Because free functional groups are located along the flexible polymer chains used in the invention methods, the metal ion can be arranged in the best position relative to the binding sites on the surface of the target molecule. As a result, the target molecule can be bound tightly, yet non-covalently, to the polymer via the multiple metal affinity complexes formed.

The existence of at least one histidine residue in the target molecule (e.g., antigen, or fusion peptide comprising a His tag), is an important factor for the binding of the antigen or therapeutic molecule to the polymer. However, with the short antigens used in the invention methods and compositions, the α-amino groups present also play a role so that in some cases the antigens can also be attached via the affinity ligand if no histidine residues are present, especially if other metal binding amino acids, such as Cysteine and Tryptophan, are present in the antigen to contribute to the binding. Since the pK value of the Histidine groups, contributing to the binding, lies in the neutral range, the binding of the antigen to the polymer might be expected to occur at a pH value of about 7. However, the actual pK value of an individual amino acid can vary strongly depending on the influence of neighboring amino acid residues. Various experiments have shown that, depending on the protein structure, the pK value of an amino acid can deviate from the theoretical pK value by up to one pH unit. Therefore, a reaction solution with a pH value of about 8 often achieves an improved binding.

Despite these complexities in the interactions taking place during formation of the metal coordination complex, the number of Histidines or Tryptophans in the antigen or target molecule provide general guidelines for selection of the metal ion to be used are found in Table 3 below:

TABLE 3

| Presence of metal binding AA | |
|---|---|
| in antigen | Suitable metal ion |
| No His or Trp | no adsorption |
| One His | Cu²⁺ |
| More than one His | Cu²⁺ or Ni²⁺ (stronger adsorption) |
| Clusters of 3 to 10 His | Cu²⁺, Ni²⁺, Zn²⁺, Co²⁺ |
| Several Trp, no His | Cu²⁺ |

pH, Buffers, and Ionic Strength

The conditions present in the reaction solution or dispersion affect formation of the metal affinity complex in the invention methods. In general, a pH value of about 8 results in stronger binding than a lower pH of about 6. Buffering agents also affect binding, with highest binding occurring in acetate or phosphate, moderate binding occurring in ammonium or Tris, and weakest binding occurring in citrate. Control of ionic strength in the reaction solution also affects complex formation. NaCl in a concentration range of about 0.1M to about 1.0 M, for example between about 0.5M and about 0.9 M may be used to suppress undesirable protein-protein ionic interactions.

The presence of other substances that also bind to the metal ions in the reaction solution or dispersion can prevent binding of the target molecule. For example, high imidazole concentrations strongly influence the binding characteristics of the metal complex, especially if the metal ion is copper. At the same time, a decrease of the pH value of the reaction solution results in adsorption of fewer of the available target molecules from a complex mixture, such as a cell lysate. In addition, to prevent ionic interactions between proteins and polymer carboxy groups that might remain uncharged with the affinity complex, relatively high ionic strength should be present. For example, the presence of about 0.1 M to 1.0 M NaCl, for example 0.5 M to about 0.9 M NaCl in the reaction solution or dispersion is sufficient to prevent undesirable protein binding in the reaction solution.

Preferably, there is at least one His at the amino- or carboxyl-terminus of the target molecule (i.e., a His tag), which results in improved specificity of binding of the antigen to the metal ion in the metal affinity complex. Therefore, in one embodiment, at least one to about 10 adjacent His residues, for example, about six His residues (i.e. His₆), are incorporated at one or both of the amino- and carboxy termini as a tag to ensure binding efficiency. If a His tag is added, the His tag and the metal chelate, for example the Ni-NTA metal chelate, are allowed to remain in the final delivery composition.

Whether or not a His tag is added to the antigen used in the invention methods, the metal coordination complex and the polymer remain along with the antigen in the vaccine delivery composition so that the antigen is non-covalently bound to the polymer via the metal coordination complex in the final product. Thus, once the coordination complex is formed linking the polymer non-covalently to the antigen, with or without the presence of a His tag, all that is required to yield the vaccine composition from the reaction solution is separation of the complex that constitutes the vaccine composition from other (i.e., unwanted) materials and proteins in the reaction solution or dispersion. A simple procedure such as size-exclusion filtration, or centrifugation and washing techniques, for example as is known in the art and described herein, can be used for this purpose.

In one embodiment, the affinity ligand-polymer composition of structural formula (III) is contained in a polymer-additional chelating agent conjugate through a linker having the structural formula (XIV), 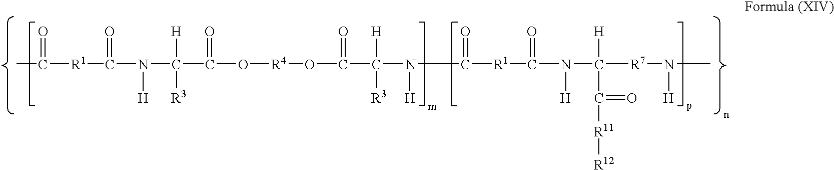
wherein R¹¹ is an optional multifunctional hydrophilic or hydrophobic linker containing 2 to 20 carbon atoms in its hydrocarbon chain, and R¹² in the metal binding ligand as shown in formula
XV. Anologous affinity ligand—polymer compositions can be prepared containing polymers of formula (V) and (VII) and ligands such as those described by Formula (XV). 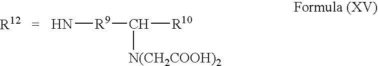
wherein, R¹⁰ is H, COOH or COOR¹³ and R¹³ is (C₁-C₈) alkyl or benzyl.

In another example, the affinity ligand 6-amino-2-(bis-carboxymethylamino)-hexanoic acid (Aminobutyl-, or AB-NTA, formula XVI): 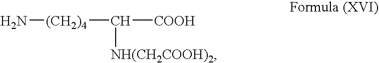
is conjugated directly, via an amide bond, to an activated carboxylate on the repeat unit of an amino acid-containing polymer, such as a PEA, PEUR or PEU. A transition metal (TM) ion as above is then bound to the chelating —NTA. In one embodiment, the resultant TM-derivatized polymer can be contacted with cell lysate for bio-functionalization via the bound TM with a genetically expressed antigen bearing a His₆ tag.

The affinity ligand (AB-NTA) of Formula XVI represents an α-N derivative of lysine. Another example of a homologous ligand disclosed herein (Example 1) is an ornithine derivative with general formula XVII. 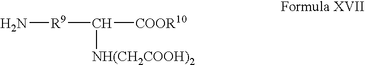
wherein R⁹ is independently (C₂-C₂₀) alkylene, (C₂-C₂₀) alkenylene; for example, (C₃-C₆) alkylene, (C₃-C₆) alkenylene; and R¹⁰ is hydrogen, (C₁-C₁₂) alkyl, or (C₂-C₁₂) alkenyl.

The complex between hexa-histidine tagged antigen or full length antigenic protein and TM-functionalized polymer can, under suitable metal affinity complex forming conditions as described herein, create cross-linked protein-polymer complexes, because only two Histidines of each hexaHis tag bind preferentially to each chelation point of the transition metal ion. Relative to lysate macromolecules, the large size of these cross-linked protein-polymer complexes, within a range controlled by stoichiometry, facilitates filtration by size-exclusion.

Alternatively, in other embodiments, an already isolated or synthetic antigen or adjuvant may be attached to the polymer via a linker molecule. Indeed, to improve surface hydrophobicity of the biodegradable polymer, to improve accessibility of the biodegradable polymer towards enzyme activation, and to improve the release profile of the biodegradable polymer, a linker may be utilized to indirectly attach the antigen and/or adjuvant to the biodegradable polymer. In certain embodiments, the linker compounds include poly(ethylene glycol) having a molecular weight (M_{w}) of about 44 to about 10,000, preferably 44 to 2000; amino acids, such as serine; polypeptides with repeat units from 1 to 100; and any other suitable low molecular weight polymers. The linker typically separates the antigen from the polymer by about 5 angstroms up to about 200 angstroms.

In still further embodiments, the linker is a divalent radical of formula W-A-Q, wherein A is (C₁-C₂₄) alkyl, (C₂-C₂₄) alkenyl, (C₂-C₂₄) alkynyl, (C₃-C₈) cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein, “alkenyl” as used to describe linkers refers to straight or branched chain hydrocarbon groups having one or more carbon-carbon double bonds.

As used herein, “alkynyl” as used to describe linkers refers to straight or branched chain hydrocarbon groups having at least one carbon-carbon triple bond.

As used herein, “aryl” as used to describe linkers refers to aromatic groups having in the range of 6 up to 14 carbon atoms.

In certain embodiments, the linker may be a polypeptide having from about 2 up to about 25 amino acids. Suitable peptides contemplated for use include poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, poly-L-lysine-L-tyrosine, and the like.

In one embodiment of the present invention, the synthetic antigen or therapeutic biologicis presented as retro-inverso or partial retro-inverso peptide.

In other embodiments the antigen is mixed with a photocrosslinkable version of the polymer in a matrix, and after crosslinking the material is dispersed (e.g. ground) to a size appropriate for uptake by a relevant antigen presenting cell or B lymphocyte, typically, but not limited to, the size range of about. 0.1-10 μm.

The linker, other than a metal affinity ligand, can be attached first to the polymer or to the antigen or adjuvant. During synthesis, the linker can be either in unprotected form or protected from, using a variety of protecting groups well known to those skilled in the art. In the case of a protected linker, the unprotected end of the linker can first be attached to the polymer or the antigen. The protecting group can then be de-protected using Pd/H₂ hydrogenolysis, mild acid or base hydrolysis, or any other common de-protection method that is known in the art. The de-protected linker can then be attached to the antigen, adjuvant, or adjuvant/antigen conjugate.

An exemplary synthesis of a biodegradable polymer according to the invention (wherein the molecule to be attached is an aminoxyl) is set forth as follows. A polyester can be reacted with an amino substituted N-oxide free radical (aminoxyl) bearing group, e.g., 4-amino-2,2,6,6-tetramethylpiperidine-1-oxy, in the presence of N,N′-carbonyldiimidazole to replace the carboxylic acid moiety at the chain end of the polyester with an amide bond to the amino substituted aminoxyl-containing radical, so that the amino moiety covalently bonds to the carbon of the carbonyl residue of the carboxyl group of the polymer. The N,N′-carbonyl diimidazole or suitable carbodiimide converts the hydroxyl moiety in the carboxyl group at the chain end of the polyester into an intermediate product moiety that will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyl diimidazole to aminoxyl is preferably about 1:1.

In such an embodiment, a typical reaction is as follows. A polyester is dissolved in a reaction solvent and reaction is readily carried out at the temperature utilized for the dissolving. The reaction solvent may be any in which the polyester will dissolve. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or dimethylsulfoxide (DMSO) at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, methylene chloride and chloroform at room temperature to 50° C. suitably dissolve the polyester.

Polymer/Antigen Linkage

In other embodiment, the polymers used to make the invention delivery compositions as described herein can have the affinity ligand, antigen, adjuvant or therapeutic biologic directly linked to the polymer. The residues of the polymer can be linked to the residues of the one or more such molecules. For example, one residue of the polymer can be directly linked to one residue of the affinity ligand. The polymer and the affinity ligand can each have one open valence. Alternatively, more than one antigen, multiple antigens, or a mixture of antigens from different pathogenic organisms can be directly linked to the polymer or can be linked to the polymer via an affinity ligand complex as described herein. However, since the residue of each antigen can be linked to a corresponding residue of the polymer, the number of residues of the one or more antigens can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of an antigen. Additionally, any synthetically feasible functional group (e.g., carboxyl) can be created on the polymer (e.g., on the polymer backbone or pendant group) to provide the open valence, provided bioactivity is substantially retained when the radical is attached to a residue of an antigen. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer of formulas (I) and (III-VII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of an antigen. Additionally, any synthetically feasible functional group (e.g., carboxyl) can be created on the compound of formulas (I) and (III-VII) (e.g., on the polymer backbone or pendant group) to provide the open valance, provided bioactivity is substantially retained when the radical is attached to a residue of an antigen. Based on the linkage that is desired, those skilled in the art can select suitably functionalized starting materials that can be derived from the compound of formula (I) and (III-VII) using procedures that are known in the art.

For example, the residue of an antigen or adjuvant can be linked to the residue of a compound of structural formulas (I) and (III-VII) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆) alkyl. Such a linkage can be formed from suitably functionalized starting materials using synthetic procedures that are known in the art. Based on the linkage that is desired, those skilled in the art can select suitably functional starting material that can be derived from a residue of a compound of any one of structural formulas (I) and (III-VII) and from a given residue of an antigen or adjuvant using procedures that are known in the art. The residue of the antigen or adjuvant can be linked to any synthetically feasible position on the residue of a compound of any one of structural formulas (I) and (III-VII). Additionally, the invention also provides compounds having more than one residue of an antigen or adjuvant bioactive agent directly linked to a compound of any one of structural formulas (I) and (III-VII).

The number of antigens or therapeutic biologics that can be linked to the polymer molecule can typically depend upon the molecular weight of the polymer. For example, for a compound of structural formulas (I) or (III), wherein n is about 5 to about 150, preferably about 5 to about 70, up to about 150 antigens (i.e., residues thereof) can be linked to the polymer (i.e., residue thereof) by reacting the antigen or an affinity ligand with end groups of the polymer. In unsaturated polymers, the antigens or affinity ligands can also be reacted with double (or triple) bonds in the polymer.

The invention delivery compositions, once formed as described herein, can be further formulated into particles. In certain embodiments, the invention vaccine delivery composition described herein can be provided as particles, with antigen/adjuvant conjugate, or antigens, with or without adjuvant, either physically incorporated (dispersed) within the particle or attached to polymer functional groups, optionally by use of a linker, using any of several techniques well known in the art and as described herein. For vaccine delivery compositions, the particles are sized for uptake by APCs, having an average diameter, for example, in the range from about 10 nanometers to about 1000 microns, or in the range from about 10 nanometers to about 100 microns. Optionally, the particles can further comprise a thin covering of the polymer to aid in control of their biodegradation. Typically such particles include from about 1 to about 150 antigens and/or adjuvant molecules per polymer molecule.

Adjuvants may be bound to the polymer covalently, bound non-covalently, or matrixed in the polymer (rather than bound). Thus, the adjuvant can be “dispersed” in the polymer of the invention composition. The method used to disperse the adjuvant in the polymer may be the same or different from the method used to attach antigen and may occur either prior to or after formation of the invention composition into particles. The method chosen will be influenced by the nature of the adjuvant. For example, an adjuvant that contains amino acids and/or a metal-binding tag can be non-covalently tethered to a polymer-affinity ligand-metal ion composition using the methods described herein for attachment of the antigen. Alternatively, a macromolecular biologic as adjuvant (or aggregates, oligomers or crystals thereof) may be covalently attached to polymer and incorporated into polymer particles so as to maintain its native activity using methods described in co-pending U.S. application Ser. No. ______ (Docket No. MEDIV3020-2), filed Nov. 21, 2006. Alternatively still, a non-polymeric adjuvant, such as an organic molecule, can be dispersed in polymer particles using methods described in co-pending U.S. application Ser. No. 11/345,021 (Docket No. MEDIV 2050-4), filed Jan. 31, 2006.

Particles of the invention delivery compositions can be made using immiscible solvent techniques. Generally, these methods entail the preparation of an emulsion of two immiscible liquids. A single emulsion method can be used to make particles that incorporate hydrophobic adjuvants. In this method, adjuvant molecules to be incorporated into the particles are mixed with polymer in solvent first, and then emulsified in water solution with a surface stabilizer, such as a surfactant. In this way, polymer particles with hydrophobic adjuvant, antigen, or adjuvant/antigen conjugates are formed and suspended in the water solution, in which hydrophobic conjugates in the particles will be stable without significant elution into the aqueous solution, but such molecules will elute into body tissue, such as muscle tissue.

Many emulsification techniques will work in making the emulsions used in manufacture of the particles. However, the presently preferred method of making the emulsion is by using a solvent that is not miscible in water. The emulsifying procedure consists of dissolving the polymer-affinity ligand complex with the solvent, mixing with any desired adjuvant molecule(s), putting into water, and then stirring with a mixer and/or ultra-sonicator. Particle size can be controlled by controlling stir speed and/or the concentration of polymer-affinity ligand complex, adjuvant molecule(s), and surface stabilizer. Coating thickness can be controlled by adjusting the ratio of the second to the third emulsion. In any of the methods of particle formation described above, the optional adjuvant can be present in a coating on the surface of the particles by conjugation to the polymers in the particles after particle formation.

Suitable emulsion stabilizers may include nonionic surface active agents, such as mannide monooleate, dextran 70,000, polyoxyethylene ethers, polyglycol ethers, and the like, all readily commercially available from, e.g., Sigma Chemical Co., St. Louis, Mo. The surface active agent will be present at a concentration of about 0.3% to about 10%, preferably about 0.5% to about 8%, and more preferably about 1% to about 5%.

The PEA, PEUR and PEU polymers described herein readily absorb water (5 to 25% w/w water up-take, on polymer film), allowing hydrophilic molecules, such as antigens and many adjuvants, to readily diffuse through them. This characteristic makes PEA, PEUR and PEU polymers suitable for use as an over coating on the polymer particles to control release rate of the antigen/adjuvant(s). Water absorption also enhances biocompatibility of the polymers and the delivery composition based on such polymers. In addition, due to the hydrophilic properties of the PEA, PEUR and PEU polymers, when delivered in vivo the particles become sticky and agglomerate, particularly at in vivo temperatures. Thus the polymer particles spontaneously form polymer depots when injected subcutaneously or intramuscularly or delivered transdermally for local delivery, such as by subcutaneous needle or needle-less injection.

Particles with average diameter range from about 1 micron to about 100 microns, which are of a size that will not permit circulation in the body, are suitable for forming such polymer depots in vivo. Alternatively, for oral administration, the GI tract can tolerate much larger particles, for example micro particles of about 1 micron up to about 1000 microns average diameter.

For instance, typically, the polymer depot will degrade over a time selected from about twenty-four hours, about seven days, about thirty days, or about ninety days, or longer. Longer time spans are particularly suitable for providing an implantable vaccine delivery composition that eliminates the need to repeatedly inject the vaccine to obtain a suitable immune response.

Rate of release of the adjuvant/antigen from the polymer particles described herein can be controlled by adjusting the coating thickness, number of adjuvant molecules covering the exterior of the particle, particle size, structure, and density of the coating. Density of the coating can be adjusted by adjusting loading of the adjuvants, if any, in the coating. When the coating contains no adjuvant, the polymer coating is most dense, and the antigen elutes through the coating most slowly. By contrast, when adjuvant/antigen is loaded into the coating, the coating becomes porous once the adjuvant/antigen has eluted out, starting from the outer surface of the coating and, therefore, the adjuvant/antigen at the center of the particle can elute at an increased rate. The higher the adjuvant loading in the covering, the lower the density of the coating layer and the higher the elution rate. The loading of adjuvant/antigen in the coating can be lower than that in the interior of the particles beneath the exterior coating. Release rate of adjuvant/antigen from the particles can also be controlled by mixing particles with different release rates prepared as described above.

In yet further embodiments, the particles can be made into nanoparticles having an average diameter of about 20 nm to about 500 nm. The nanoparticles can be made by the single emulsion method with the antigen dispersed therein, i.e., mixed into the emulsion or conjugated to polymer as described herein. The nanoparticles can also be provided as micelles containing the PEA or PEUR polymers described herein. The micelles are formed in water and the water soluble antigens with optional adjuvant protein are loaded into micelles at the same time without solvent.

More particularly, the biodegradable micelles are formed of a water soluble ionized polymer chain conjugated to a hydrophobic polymer chain. Whereas, the outer portion of the micelle mainly consists of the water soluble ionized section of the polymer, the hydrophobic section of the polymer mainly partitions to the interior of the micelles and holds the polymer molecules together.

The biodegradable hydrophobic section of the polymer used to make micelles is made of PEA, PEUR or PEU polymers, as described herein. For strongly hydrophobic PEA, PEUR or PEU polymers, components such as di-L-leucine ester of 1,4:3,6-dianhydro-D-sorbitol or a rigid aromatic di-acid like α,ω-bis (4-carboxyphenoxy) (C₁-C₈) alkane may be included in the polymer repeat unit. By contrast, the water soluble section of the polymer comprises repeating alternating units of polyethylene glycol, polyglycosaminoglycan or polysaccharide and at least one ionizable or polar amino acid, wherein the repeating alternating units have substantially similar molecular weights and wherein the molecular weight of the polymer is in the range from about 10 kD to about 300 kD. The higher the molecular weight of the water soluble section, the greater the porosity of the micelle, with the longer chains enabling high loading of the water soluble antigens and optional adjuvants. In addition, polyamino acids are more immunogenic than single amino acids.

The repeating alternating units may have substantially similar molecular weights in the range from about 300D to about 700D. In one embodiment wherein the molecular weight of the polymer is over 10 kD, at least one of the amino acid units is an ionizable or polar amino acid selected from serine, glutamic acid, aspartic acid, lysine and arginine. In one embodiment, the units of ionizable amino acids comprise at least one block of ionizable poly(amino acids), such as glutamate or aspartate, can be included in the polymer. The invention micellar composition may further comprise a pharmaceutically acceptable aqueous media with a pH value at which at least a portion of the ionizable amino acids in the water soluble sections of the polymer are ionized.

The biodegradable hydrophobic polymer chain is made of PEA, PEUR or PEU polymers, as described herein. For a strongly hydrophobic PEA, PEUR or PEU, components such as 1,3-bis(-4-carboxylate-phenoxy)-propane (CPP) and/or bis(-L-leucine) diesters of -1,4:3,6-dianhydrohexitoles-D-sorbitol (DAS) may be included in the hydrophobic polymer chain. By contrast, the water soluble chain is made of many repeating units of poly-ethylene glycol (PEG) and an ionizable amino acid, such as (poly)lysine or (poly) glutamate, wherein the PEG unit and the ionizable amino acid unit have similar molecular weights, for example, a few hundred kD (i.e., the PEG unit can have a molecular weight at substantially any value in this range). However, the total molecular weight of the water soluble section of the polymer can be, for example, in the range of about 10 kD to about 300 kD. The higher the molecular weight of the water soluble section, the greater the porosity of the micelle, with the longer chains enabling high loading of the water soluble antigens and optional adjuvants. In addition, polyamino acids are more immunogenic than single amino acids.

Charged moieties within the micelles partially separate from each other in water, and create space for absorption of water soluble agents, such as the antigen-containing affinity complex attached to the polymer and optional adjuvant. Ionized chains with the same type of charge will repel each other and create more space. The ionized polymer also attracts the antigen, providing stability to the matrix. In addition, the water soluble exterior of the micelle prevents adhesion of the micelles to proteins in body fluids after ionized sites are taken by the adjuvant(s). This type of micelle has very high porosity, up to 95% of the micelle volume, allowing for high loading of aqueous-soluble biologics, such as various adjuvants. Particle size range of the micelles is about 20 nm to about 200 nm, with about 20 nm to about 100 nm being preferred for circulation in the blood.

Rate of release of the adjuvant/antigen from the polymer particles described herein can be controlled by adjusting the coating thickness, particle size, structure, and density of the coating. Density of the coating can be adjusted by varying the loading of the adjuvant/antigen in the coating. When the coating contains no antigen or adjuvant, the polymer coating is densest, and the elution of the antigen and optional adjuvant through the coating is slowest. By contrast, when antigen or adjuvant is loaded into the coating, the coating becomes porous once the antigen or adjuvant has eluted out, starting from the outer surface of the coating and, therefore, the active agent(s) at the center of the particle can elute at an increased rate. The higher the loading in the coating layer, the lower the density and the higher the elution rate. The loading of adjuvant/antigen in the coating can be lower than that in the interior of the particles beneath the exterior coating. Release rate of adjuvant/antigen from the particles can also be controlled by mixing particles with different release rates prepared as described above.

Particle size can be determined by, e.g., laser light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM). In order to do so, dry particles are sputter-coated with a gold/palladium mixture to a thickness of approximately 100 Angstroms, and then examined using a scanning electron microscope. Alternatively, the antigen, rather than being non-covalently attached to the polymer via the antigen-containing affinity complex, can be dispersed in the polymer (i.e., by “loading” or “matrixing”), using any of several methods well known in the art and as described hereinbelow. The antigen content is generally in an amount that represents approximately 0.1% to about 40% (w/w) antigen to polymer, for example, about 1% to about 25% (w/w) antigen, or about 2% to about 20% (w/w) antigen. The weight percentage of antigen will depend on the desired dose and the condition being treated, as discussed in more detail below. In any event, following preparation of the invention delivery compositions, whether as particles or polymer molecules, the composition can be lyophilized and the dried composition suspended in an appropriate vehicle prior to use.

Any suitable and effective amount of particles or polymer fragments containing the antigen and any adjuvant or therapeutic biologic included in the invention delivery compositions can be released with time from the polymer particles (including those in a polymer depot formed in vivo) and will typically depend, e.g., on the specific polymer, antigen, adjuvant or therapeutic biologic used as well as polymer/antigen linkage, if present. Typically, up to about 100% of the polymer particles or molecules can be released from the polymer depot. Specifically, up to about 90%, up to 75%, up to 50%, or up to 25% thereof can be released from the polymer depot. Factors that typically affect the release rate from the polymer are the nature and amount of the polymer, the types of polymer/antigen linkage and/or polymer/therapeutic biologic linkage, and the nature and amount of additional substances present in the formulation.

Once the delivery compositions is assembled using the invention methods, as above, the composition can be formulated for subsequent delivery. For example, for mucosal or subcutaneous delivery, the compositions will generally include one or more “pharmaceutically acceptable excipients or vehicles” appropriate for mucosal or subcutaneous delivery, such as water, saline, glycerol, polyethyleneglycol, hyaluronic acid, ethanol, etc. Additionally, auxiliary substances, such as wetting or emulsifying agents, pH buffering substances, and the like, may be present in such vehicles.

Intranasal and pulmonary formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption by the nasal mucosa.

For rectal and urethral suppositories, the vehicle will include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the formulations of the present invention can be incorporated in pessary bases, such as those including mixtures of polyethylene triglycerides, or suspended in oils such as corn oil or sesame oil, optionally containing colloidal silica. See, e.g., Richardson et al., Int. J. Pharm. (1995) 115:9-15.

For a further discussion of appropriate vehicles to use for particular modes of delivery, see, e.g., Remington: The Science and Practice of pharmacy, Mack Publishing Company, Easton, Pa., 19th edition, 1995. One of skill in the art can readily determine the proper vehicle to use for the particular antigen and site of delivery.

The compositions assembled in the invention methods may comprise an “effective amount” of the antigen or therapeutic biologic of interest. That is, an amount of antigen will be included in the compositions that will cause the subject to produce a sufficient immunological response in order to prevent, reduce or eliminate symptoms. Alternatively, an amount of therapeutic biologic will be included in the compositions that will prevent, reduce or eliminate symptoms. The exact amount necessary will vary, depending on the subject being treated; the age and general condition of the subject to be treated; the capacity of the subject's immune system to synthesize antibodies or an appropriate cell-mediated response; the degree of protection desired; the severity of the condition being treated; the particular antigen or therapeutic biologic selected and its mode of administration, among other factors. An appropriate effective amount can be readily determined by one of skill in the art. Thus, an “effective amount” will fall in a relatively broad range that can be determined through routine trials. For example, for purposes of the present invention, an effective dose will typically range from about 1 μg to about 100 mg, for example from about 5 μg to about 1 mg, or about 10 μg to about 500 μg of the antigen delivered per dose.

Once formulated, the compositions of the invention are administered mucosally or subcutaneously by injection, or by other delivery route, using standard techniques. See, e.g., Remington: The Science and Practice of pharmacy, Mack Publishing Company, Easton, Pa., 19th edition, 1995, for mucosal delivery techniques, including intranasal, pulmonary, vaginal and rectal techniques, as well as European Publication No. 517,565 and Illum et al., J. Controlled Rel. (1994) 29:133-141, for techniques of intranasal administration.

Dosage treatment may be a single dose of the invention time release delivery composition, or a multiple dose schedule as is known in the art. For vaccine delivery compositions, a booster may be with the same formulation given for the primary immune response, or may be with a different formulation. The dosage regimen will also be determined, at least in part, by the needs of the subject and be dependent on the judgment of the practitioner. Furthermore, if prevention of disease is desired, the vaccine delivery composition is generally administered prior to primary infection with the pathogen of interest. If treatment is desired, e.g., the reduction of symptoms or recurrences, the vaccine delivery compositions are generally administered subsequent to primary infection.

The invention compositions can be tested in vivo in a number of animal models developed for the study of subcutaneous or mucosal delivery. For example, the conscious sheep model is an art-recognized model for testing nasal delivery of substances. See, e.g., Longenecker et al., J. Pharm. Sci. (1987) 76:351-355 and Illum et al., J. Controlled Rel. (1994) 29:133-141. The vaccine delivery composition, generally in powdered, lyophilized form, is blown into the nasal cavity. Blood samples can be assayed for antibody titers using standard techniques, known in the art, as described above. Cellular immune responses can also be monitored as described above.

There are currently a series of in vitro assays for cell-mediated immune response that use cells from the donor, which may be either an immunized human volunteer who donates blood, or a mouse or other animal. The assays include situations where the cells are from the donor, however, some assays provide a source of antigen presenting cells from other sources, e.g., B cell lines. These in vitro assays include cell surface marker analysis by fluorescence activated flow cytometry, assays for cytokine production such as the intracellular cytokine assay, and the enzyme-linked immunosorbent spot assay (ELISPOT), analysis of antigen-specific T cell receptor expression (tetramer analysis by flow cytometry), the cytotoxic T lymphocyte assay; lymphoproliferative assays, e.g., tritiated thymidine incorporation; the protein kinase assays, the ion transport assay and the lymphocyte migration inhibition function assay (Hickling, J. K. et al. (1987) J. Virol., 61: 3463; Hengel, H. et al. (1987) J. Immunol., 139: 4196; Thorley-Lawson, D. A. et al. (1987) Proc. Natl. Acad. Sci. USA, 84: 5384; Kadival, G. J. et al. (1987) J. Immunol., 139:2447; Samuelson, L. E. et al. (1987) J. Immunol., 139:2708; Cason, J. et al. (1987) J. Immunol. Meth., 102:109; and Tsein, R. J. et al. (1982) Nature, 293: 68.

To test whether a peptide recognized by a T cell will activate the T cell to generate an immune response, a so-called “functional test” is used. The enzyme-linked immunospot (ELISpot) assay has been adapted for the detection of individual cells secreting specific cytokines or other effector molecules by attachment of a monoclonal antibody specific for a cytokine or effector molecule on a microplate. Cells stimulated by an antigen are contacted with the immobilized antibody. After washing away cells and any unbound substances, an enzyme tagged polyclonal antibody or more often, a monoclonal antibody, specific for the same cytokine or other effector molecule is added to the wells. Following a wash, a substrate for the tagged antibody is added under reactive conditions such that a colored precipitate (or spot) forms at the sites of cytokine localization. The spots can be counted manually or with automated ELISpot reader composition to quantitate the response. A final confirmation of T cell activation by the test peptide may require in vivo testing, for example in a mouse or other animal model.

As is readily apparent, the vaccine delivery compositions assembled using the invention methods are useful for eliciting an immune response against viruses, bacteria, parasites and fungi, for treating and/or preventing a wide variety of diseases and infections caused by such pathogens, as well as for stimulating an immune response against a variety of tumor antigens. Not only can the compositions be used therapeutically or prophylactically, as described above, the compositions may also be used in order to prepare antibodies, both polyclonal and monoclonal, for, e.g., diagnostic purposes, as well as for immunopurification of the antigen of interest. If polyclonal antibodies are desired, a selected mammal, (e.g., mouse, rabbit, goat, horse, etc.) is immunized with the compositions of the present invention. The animal is optionally boosted 2-6 weeks later with one or more administrations of the antigen. Polyclonal antisera is then obtained from the immunized animal and treated according to known procedures, for example, to determine whether a protective or therapeutic response has been elicited. See, e.g., Jürgens et al. (1985) J. Chrom. 348:363-370.

Monoclonal antibodies are generally prepared using the method of Kohler and Milstein, Nature (1975) 256:495-96, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent T cells) by applying a cell suspension to a plate or well coated with the protein antigen. B cells, expressing membrane-bound immunoglobulin specific for the antigen, will bind to the plate, and are not rinsed away with the rest of the suspension. Resulting B cells, or all dissociated spleen cells, are then induced to fuse with myeloma cells to form hybridomas, and are cultured in a selective medium (e.g., hypoxanthine, aminopterin, thymidine medium, “HAT”). The resultant hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (as ascites in mice). See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,121; 4,427,783; 4,444,887; 4,466,917; 4,472,500, 4,491,632; and 4,493,890. Panels of monoclonal antibodies produced against the polypeptide of interest can be screened for various properties; i.e., for isotype, epitope, affinity, and the like.

The following examples are meant to illustrate, and not to limit, the invention.

### EXAMPLE 1

Synthesis of Affinity Ligands:

Synthesis of three ligands useful for metal complex formation is here described.

The affinity ligand 6-amino-2-(bis-carboxymethylamino)-hexanoic acid (AB-NTA), (Formula XVI), was synthesized according to published procedure (E. Hochuli, H. Döbeli and A. Schacher J. Chromatography, 411, 177-184, 1987).

NTA(Orn)-Ligand Synthesis (Formula XVII, wherein R⁹═(CH₂)₃; and R¹⁰=H)

N^{δ}-Z-NTA(Orn)-N-alkylation step: 4.17 g Bromoacetic acid (30.0 mmol) was dissolved in 15 mL of 1.5 N NaOH and cooled to 0° C. 3.99 g of N^{δ}-Benzyloxycarbonyl-L-ornithine (15.0 mmol) in 25 mL of NaOH was added dropwise to this solution. Initially, the solution became milky white, but after 5.0 mL of 1.5 N NaOH was added, the solution turned clear again. After 2 hours the cooling bath, the solution was stirred overnight at room temperature (pH was maintained around ˜12.0 or above, otherwise precipitate was formed). After heating at 50° C. for 2 hours and cooling to room temperature, 60 mL of 1M HCl was added dropwise. Formed precipitate was filtered over a centered funnel. The white solid so obtained was rinsed with DI water (2×25 mL) and dried in the vacuum at 45° C. Pure product yield was 2.9 g.

NTA(Orn)-Hydrogenation step: N^{δ}-Z-NTA(Orn) (2.5 g, 6.53 mmol) was dissolved in 66 mL of methanol/water (20:1) and, after the addition of 125 mg of 10% Pd/C (˜5% by weight), was hydrogenated at room temperature and atmospheric pressure. The hydrogenation was completed in 2.5 hours as monitored by TLC in CH₃CN/H₂O (4:1) developed with UV and ninhydrin. The catalyst was removed over a celite bed and the organic solvent was evaporated in vacuo. Lyophilized product was collected.

Preparation of Affinity Ligand AB-NTA-OMe (Formula XVII, wherein R⁹=(CH₂)₄; and R¹⁰=CH₃)

In the first stage, 1.5 g of 5-(Bis-benzyloxycarbonylmethyl-amino)-5-methoxycarbonyl-pentyl-ammonium chloride (Formula XVI) was synthesized based on a reported procedure (Kiessling L L et al., J. Am. Chem. Soc., (2004), 126, 1608-1609). The phenyl-protected ligand is designated as NTA-OMe-(CO₂CH₂Ph)₂. Attachment of the ligand to PEA and further deprotection is described below in Example 2.

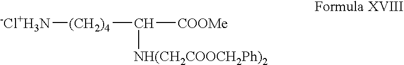

### EXAMPLE 2

General Procedure for Activation of PEA (PEA-OSu)

24.0 g (13.09 mmol, weight average Mw=65 kDa, GPC (PS)) of PEA polymer (Formula III; wherein R¹=(CH₂)₈; R²═H; and R³═CH₂CH(CH₃)₂), was dissolved in 80 mL dry dimethylformamide (DMF) under argon. Then, 2.97 g dicyaclohexylcarbodiimide (DCC, 1.1 eq, 14.41 mmol) and 1.81 g N-hydoxysuccinimide (HOSu, 15.71 mmol) were separately dissolved in DMF (5-10 mL) and added to the solution 10 minutes apart. The reaction mixture was allowed to stir for about 24 hours at room temperature. Formed residue was removed by filtering through 0.45 micron pore size frit (PTFE filters). A solution of PEA-OSu conjugate was collected into another 1.0 L round bottom flask and kept under argon. 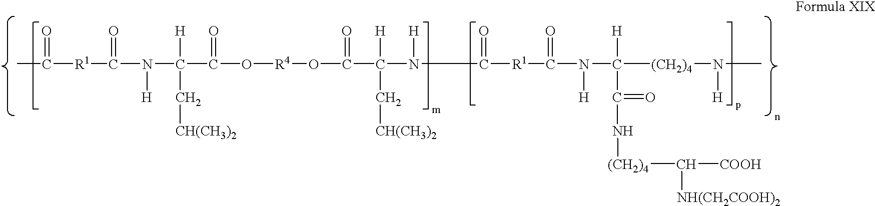
Synthesis of PEA-NTA Conjugate (Formula XIX)

3.43 g (13.09 mol) of 6-amino-2-(bis-carboxymethylamino)-hexanoic acid was stirred in 60 mL dimethylsulfoxide (DMSO) and added 7.53 mL of diisopropylethylamine (DIPEA) (3.3 eq. 43.21 mmol). The resulted heterogeneous mixture was diluted with additional 40 mL of DMF and mixed on a vortex mixer certain glycolipids, membrane lipids or nucleic acids, at room temperature for 5 minutes. The NTA salt dispersion formed was added slowly to the above activated ester of PEA-OSu (24.0 g, 13.09 mmol) in a 1.0 L round bottom flask. The resulting reaction mixture was stirred for 72 hours at room temperature (NTA consumption was monitored by TLC, Ninhydrin spray and ¹H NMR). PEA-NTA polymer conjugate was precipitated into a 1 L of 0.1 N HCl solution and was kept stirring for one hour. The precipitate was collected by filtration, cut into small pieces, and washed twice with 500 mL de-ionized water for one hour. Polymer conjugate dried overnight in a lyophilizer, (crude yield 25.2 g.). The obtained polymer was further purified by dissolving in ethanol (5 g in 40 mL) and precipitating into 0.7 L of water. After one hour of vigorous stirring, formed precipitate was collected, cut into small pieces, placed in 1.0 L of deionized water and stirred for another hour. The polymer was collected and dried overnight in a vacuum oven at 45° C. Formed solid was redissolved into ethanol, filtered and placed on a Teflon® treated dish. After drying in the vacuum oven, product was analyzed by NMR and GPC and tested for traces of HCl and DIPEA.

Synthesis of PEA-NTA(Orn)-Conjugate

The ornithine analog was similarly synthesized. In a 8 mL vial, 0.137 g of NTA(Orn) (1.0 eq, 0.55 mmol) was dissolved in 3.0 mL DMSO and 0.32 mL of DIPEA was added to the solution (3.3 eq., 1.82 mmol). The resulting heterogeneous mixture was vortexed and stirred at room temperature for 5 minutes (another 0.6 mL of DMF was added to aid dispersal). NTA(Orn) salt suspension formed in DMSO-DMF was added slowly to the activated ester of PEA-OSu (65 k) (1.02 g, 0.55 mmol) in 20 mL vial under argon and stirred for 72 hours at room temperature. NTA(Orn) consumption was monitored by TLC, Ninhydrin spray and ¹H NMR. Polymer from the reaction mixture was precipitated in 150 mL, 1.0 N HCl, under vigorous stirring. Collected polymer was cut into small pieces and allowed to stir for one hour. Finally, polymer pieces were placed in 0.2 L D water and stirred for one hour to remove the traces of HCl (this process was repeated two times). Polymer pieces were collected and dried overnight in a lyophilizer (Yield: 1 g.).

PEA-NTA(OMe) Conjugation/Deprotection:

PEA-NTA-OMe-(CO₂CH₂Ph)₂ conjugation Conjugation of PEA-NTA(OMe) to activated PEA-OSu was conducted analogous to two previous procedures. Formed PEA-ligand conjugate was further deprotected as follows: In a 100 mL round bottom flask, 250 mg of solid PEA-NTA-OMe-(CO₂CH₂Ph)₂ was placed in 10 ml of ethanol. After complete dissolution, 1.0 mL of formic acid and 25-30 mg of 10% Pd/C were added and the flask was purged with argon and stirred overnight. The next day, the reaction mixture was filtered through 0.45 micron pore size PTFE frit and rinsed with additional 4.0 mL of ethanol. The total mixture was added in 30 mL D.I. water and polymer was precipitated as a white solid. The solid was cut into small pieces and stirred in 20 mL of D.I. water for 30 minutes (repeated two times). The pieces were dried in the oven for 24 hours and yielded 230 mg of the product.

### EXAMPLE 3

Preparation of PEA-NTA-Ni²⁺ Complex

To a solution of 2.3 g of PEA-NTA in ethanol (44 mL, a solution of NiCl₂ in DI water (118 mg in 40 mL) was added dropwise under sonication. Polymer-NTA-Ni²⁺ complex was slowly precipitated as a greenish solid. The heterogeneous mixture was kept at room temperature for one hour and sonicated every 15 minutes in 30 second bursts. After centrifugation and decantation, the PEA-NTA-Ni²⁺ complex was washed with DI water (3×40 ml) and lyophilized. Dried PEA-NTA-Ni²⁺ complex was dissolved in methanol (60 ml) and cast on a Teflon® treated dish. After complete evaporation of methanol at room temperature, the drying was continued at 40-45° C. in a vacuum oven for 48 hrs. The yield of the complex was 94.2% (2.278 g).

### EXAMPLE 4

Procedure for the Assembly of Invention Vaccine Delivery Compositions from His-Tagged Proteins

A. Preparation of a stock solution in which a nickel affinity ligand is conjugated to PEA. PEA-NTA-Ni⁺² stock solution A was prepared as follows: 101.9 mg of nickelated polymer (weight average Mw=68.9 kDa) was placed in a vial in 2.4 mL of hexafluoroisopropanol (HFIP). Resultant heterogeneous mixture was sonicated and left at room temperature for two hours to soak until it became a gel. Thereafter, 2.0 ml of D.I. water was added drop wise to formulate a fine dispersion (with pH of 3.0) of the gel.

B. Preparation of a stock solution in which an antigen protein is captured by metal-loaded NTA-PEA matrix: 2.15 mL of stock solution A (which contained 49.89 mg PEA-NTA-Ni⁺²) was added drop-wise to a chilled solution (at about 4° C.) of 21.0 mg of purified His-Tagged E6E7 Protein (SEQ ID NO:17, the target antigen for the HPV therapeutic vaccine)in 30 mL buffer (25 mM Tris/500 mM NaCl). The precipitation of protein-polymer metal affinity complex started within minutes at pH 8.0. The resultant mixture was allowed to stay at the same temperature for an hour to ensure complete precipitation of protein and polymer. The precipitate was collected by centrifugation at 12000 rpm at +4° C. for 30 minutes. (Supernatant was collected in a separate tube and analyzed with SDS PAGE for any remaining protein). Precipitate was rinsed twice with 30 mL of PBS buffer, followed by centrifugation at 12000 rpm at 4° C. for 30 minutes. Finally, the collected light green colored precipitate was lyophilized for 24 hours. This process yielded 66 mg of formulation (with 95% yield of protein). Protein capture in the formulation was analyzed by reducing SDS PAGE, as well as by other methods.

Ground Formulation of PEA-NTA-E6E7

A complex of 29.18 mg of PEA-NTA-Ni⁺²-E6E7 protein was formed as follows. 6.5 mg of His₆ tagged E6E7 protein (SEQ ID NO: 17) was suspended in 6.5 ml of PBS buffer. This material was ground in a tissue grinder for 10 to 15 minutes to achieve a uniform dispersion.

### EXAMPLE 5

Procedure for the Assembly of Pre-Fabricated Vaccine Delivery Particles

A) Formulation of PEA-NTA-Ni⁺² Microspheres with in-situ nickelation PEA-NTA-Ni⁺² microparticles were prepared by dissolving 50 mg of PEA-NTA (formed in Example 3 above) in 1 mL hexafluoroisopropanol (HFIP) over 5 minutes of sonication at room temperature. An aqueous in organic emulsion was generated when 250 μL of 0.1 M NiSO₄ was added to the PEA-NTA/HFIP phase. The emulsion was rendered homogeneous by subsequent addition of 750 μL HFIP and 500 μL D.I. water, while vortexing the entire emulsion for 5 minutes to form “phase 1”. A secondary organic/aqueous in aqueous emulsion was generated when phase 1 was injected into “phase 2”, which consisted of poly(vinyl) alcohol (PVA) in D.I. water (25 mg of PVA in 12 mL D.I. water). Phase 1 was injected into phase 2 via a 20 gauge needle during ultrasonication, 25 W of power, over 60 seconds at 10° C. The resultant emulsion, “phase 3”, was rotoevaporated at 760 mmHg vaccum for 10 minutes in a 30° C. bath to remove the organic solvent, resulting in a solution of PEA-NTA microspheres. This microsphere solution was filtered through a 0.001″ stainless steal mesh, frozen in liquid nitrogen, and lyophilized overnight.

B) Formulation of PEA-NTA-Ni⁺² Microspheres with pre-nickelation PEA-NTA-Ni⁺² microparticles were prepared with the pre-nickelated PEA-NTA-Ni⁺² complex from Example 3 above by dissolving 50 mg of the complex in 1 mL hexafluoroisopropanol (HFIP) over 5 minutes of sonication at room temperature. The solution was rendered homogenous with the addition of 600 μL D.I. water, while vortexing the emulsion for 5 minutes to form “phase 1”. An organic in aqueous emulsion was formed by injecting phase 1 into “phase 2”, which consited of poly(vinyl) alcohol (PVA) dissolved in D.I. water (7 mg of PVA in 25 mL D.I. water). Phase 1 was injected into phase 2 via a 20 gauge needle at 110° C. to form a “phase 3” emulsion. The phase 3 emulsion was ultrasonicated with 25 W of power, over 60 seconds at 10° C., then rotoevaporated at 760 mmHg vaccum for 10 minutes in a 30° C. bath to remove the organic solvent, filtered through a 0.001″ stainless steal mesh to form PEA-NTA-Ni⁺² microspheres, frozen in liquid nitrogen, and lyophilized overnight.

C) Assembly of His-Tagged Proteins onto Pre-Fabricated PEA-NTA-Ni⁺² Microspheres Microspheres from either (A) or (B) described in Example 5 were reconstituted in purified antigen solutions at concentrations ranging from 1-3 mg per mL. Typical particle diameters ranged from 0.05-15 μm. For example, 5 mg of purified Histidine-tagged E6E7 protein were coupled to 20 mg of these PEA-NTA-Ni⁺² microspheres by reconstitution of the particles in 10 mL of the purified E6E7 protein solution (TRIS pH 8.0 buffer) with pipet mixing. This method of pre-fabrication of the nicelated microspheres avoids exposure of the His-tagged proteins to sonication or organic solvents, as is was done in formation of the invention compositions whose fabrication is described in Example 4. This aspect of the method can be important for antigens in which important conformational antigenic determinants can be disrupted in certain solvents, for example, the influenza hemagglutinin described in Example 10.

### EXAMPLE 6

This example illustrates the use in animals of PEA polymer in the invention vaccine delivery composition, with or without additional adjuvants. A modified fusion protein based on the E6 and E7 proteins of human papillomavirus (HPV) subtype 16 (SEQ ID NO:17) was used as the antigen in the model system described below.

Experiments were carried out on female C57BL/6 mice between 6-10 weeks of age, purchased from Taconic (Hudson N.Y.). The subunit vaccine consisted of His₆ tagged-E6E7 fusion protein produced as a recombinant molecule in E. coli, complexed to microspheres of PEA-NTA-Ni⁺² as described in Example 4. This material was diluted in saline solution, or in saline containing the adjuvant CpG at a final concentration of 5 nmol (31.5 μg) CpG per animal. The amount of E6E7 protein used per dose was between 10-100 μg, as noted in each example. The synthetic oligodeoxynucleotide CpG (5′ to 3′: tccatgacgttcctgatgct) (SEQ ID NO:20) was synthesized with a phosphothioate backbone by Integrated DNA Technologies (Coralville Iowa). Polymer-protein conjugate and CpG were mixed together one hour prior to immunization, and the solutions sonicated (1 min at 4° C.) immediately before injection to disperse the particles. Mice were immunized subcutaneously at the base of the tail, in a total volume of 200 μl.

The cell line C3 is a mouse embryonic fibroblast transformed with the entire HPV-16 genome as described elsewhere, (Ossevoort M A, et al. J Immunother Emphasis Tumor Immunol. (1995), 18(2): 86-94.). When injected subcutaneously on the flank of a syngeneic unimmunized mouse, a palpable tumor can be detected approximately 10 days post-injection. Prevention of tumor growth, or regression of existing tumors, is the primary assay used to determine the efficacy of each vaccine formulation.

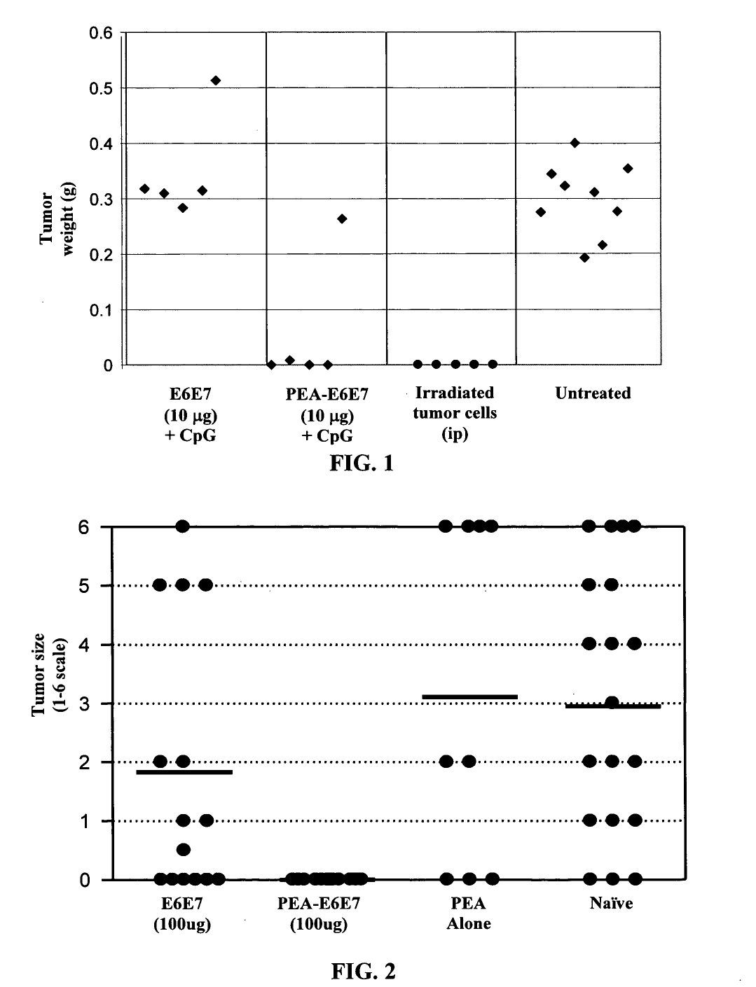

As a test of the above described PEA-NTA-Ni⁺² vaccine delivery compositions (“the vaccine”) to act prophylactically, a mouse experiment was set up to monitor prevention of tumor growth in mice immunized five weeks prior to tumor challenge. In this study, four groups of five mice were prepared as follows: Group 1) immunized with 10 μg purified above-described HPV protein antigen plus 5 nmol CpG as immunostimulatory adjuvant, Group 2) immunized with the vaccine (normalized to 10 μg protein) plus 5 nmol CpG, Group 3) injected intraperitoneally with about 1×10⁶ irradiated C3 tumor cells, (as a positive control), or Group 4) left unimmunized (naïve group). After five weeks, mice were injected subcutaneously (on the flank) with 3×10⁵ C3 tumor cells. Tumor growth was monitored over 15 days following cell injection, at which point the animals were sacrificed, and the tumors excised and weighed. As shown in FIG. 1, mice immunized with the vaccine had smaller tumors than those immunized with unconjugated HPV protein antigen, or left unimmunized (naïve).

### EXAMPLE 7

Prevention of Tumor Growth in Mice Immunized One Week Prior to Tumor Cell Challenge

Groups of 10-15 mice were either immunized with Group 1) 100 μg purified HPV protein antigen, Group 2) PEA-NTA-Ni⁺²-antigen vaccine delivery composition (“the vaccine”), prepared as described in Example 5, above) (containing 100 μg protein), Group 3) PEA polymer alone (no antigen), or Group 4) left unimmunized (naïve group). After seven days, mice were injected subcutaneously (on the flank) with 2×10⁵ C3 tumor cells. Tumor growth was monitored over 18 days following cell injection, and tumor size scored by palpation, using a scale of 1-6. As shown by data in FIG. 2, mice immunized with the vaccine were 100% protected from tumor growth, even without the use of additional adjuvant. Mice immunized with protein alone or polymer alone, or mice that were not immunized, were not protected from tumor growth.

Some mice from each group were sacrificed on the day of tumor injection, or seven days after tumor injection, and their spleens removed for analysis. Mice that received the vaccine were shown to have an elevated number of E6E7-specific CD8 T cells, and these cells were shown to produce interferon-γ (IFN-γ) in response to antigenic stimulation in vitro.

### EXAMPLE 8

Regresssion of Tumors Induced by a Therapeutic Immunization One Week after Tumor Cell Challenge.

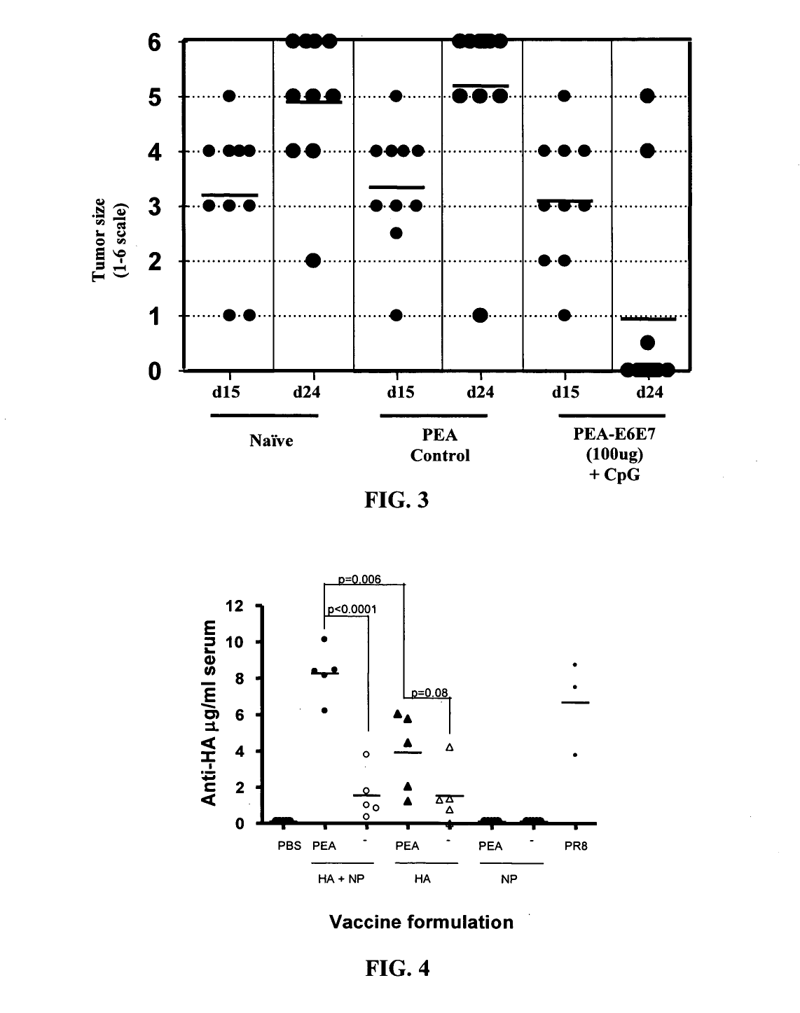

Mice were injected with 4×10⁵ C3 tumor cells subcutaneously in the flank. Six days later, groups of 5 mice were either Group 1) left unimmunized (naïve group), Group 2) PEA polymer alone (no antigen), or Group 3) the vaccine formulated as microspheres as described in Example 6 herein (normalized to 100 μg protein) plus 5 nmol CpG as adjuvant. Tumor growth was monitored over 24 days following cell injection, and tumor size scored by palpation, using a scale of 1-6. As shown in FIG. 3, tumors in mice immunized with the vaccine regressed between days 15 and 24, while tumors in unimmunized mice, or in mice immunized with PEA polymer alone, continued to grow.

### EXAMPLE 9

Expression, Purification, and Characterization of the Ectodomain of HA

Designing of Oligonucleotides Sets of overlapping oligonucleotides were designed to make gene cassettes encoding the ectodomain of hemagglutinin from Influenza A/Puerto Rico/8/34 (HAPR8) (SEQ ID NO:11). These DNA cassettes were designed as Nde1-EcoRI restriction fragments with carboxy-terminal hexa-histidine tags for purification purposes and for assembly of the vaccine composition according to the invention method. The DNA expression cassettes were designed without unwanted restriction sites and with codon usage selected for bacteria. The overlapping oligonucleotides were limited in length to 85 nucleotides to ensure high accuracy at the ends.

Cloning and Sequencing Synthetic oligonucleotides were received lyophilized and were suspended to a concentration of 100 pmol/ml. The oligonucleotides were then annealed in pairs by heating and cooling and extended in groups with the Klenow fragment of DNA polymerase I. Next, these annealed and extended sequences were joined by the polymerase chain reaction (PCR) using a high-fidelity polymerase mixture (Roche). The PCR products were then TOPO-cloned into pCR2.1 or pBAD TOPO topoisomerase-linked vectors (Invitrogen, San Diego, Calif.), transformed into TOP10 bacteria and grown on selective plates.

Four-milliliter bacterial cultures of individual colonies of TOP10 were grown and plasmid DNA was prepared. The plasmid preparations were then analyzed by restriction digestion and the DNA from positive clones was sequenced. The DNA fragment was subcloned by restriction digestion and ligation into expression vectors. For bacterial expression, two vector families were used: (1) the pBAD vectors, which drive transcription of the gene using an arabinose-inducible promoter; and (2) vectors using the T7 promoter, such as the pET vector, which requires T7 polymerase to be induced within the bacteria chosen for protein expression. The arabinose promoter has the capacity to be modulated by varying the inducer arabinose concentration in a bacterial cell strain like TOP10 that does not metabolize arabinose, while the T7 promoter is driven strongly by the presence of even a small amount of induced T7 polymerase, so one can produce a large amount of protein quickly. In addition, the HAPR8 and HA1PR8-encoding DNA cassettes were subcloned into pFAST Bac Dual vector (Invitrogen) to use to make recombinant baculovirus (Bacmid). In one example, the DNA cassette encoding the amino acids of SEQ ID NO:11 were inserted in pBac Dual in a manner that the protein expression was driven by the polyhedron promoter. The baculovirus produced from these transfected cells was called pBac-HAPR8 baculovirus.

### EXAMPLE 10

Production and Formulation of HA and Measurement of Activity

Because the conformational state of HA is critical for robust protective B cell responses, baculovirus-infected SF9 cells were selected for expression of HA and the purified HAPR8 protein was formulated in PEA-NTA-Ni⁺² microspheres. The pBac-HAPR8 baculovirus was used at a multiplicity of infection of 1 (MOI=1) to infect SF9 cells in 500 ml of Sf900 II-SFM medium (Invitrogen) at a density of 1.5×10⁶ cells per milliliter. The infected cells were grown for 48 to 72 hours and harvested by centrifugation. The cell proteins were solubilized by suspension in PBS buffer containing 0.1% Triton X-100® and protease inhibitors and purified by immobilized metal affinity chromatography using Ni-loaded chelating sepharose(GE). Purified protein was dialyzed against two changes of 50 volumes of 25 mM Tris® surfactant, pH 8.0, 150 mM NaCl, filtered through 2 micron filters and tested for endotoxin.

Characterization of the purified proteins consists of SDS-PAGE, size-exclusion chromatography, as well as immunoblotting and ELISA for reactivity. In addition, since the HA antigens must be properly folded, the HA proteins were tested for sialic acid binding function by a hemagglutination assay following standard protocols (i.e., Webster, R., et al., WHO Animal Influenza Manual, World Health Organization, WHO/CDS/NCS/2002.5). Chicken red blood cells were used in an agglutination assay with A/Puerto Rico/8/34 virus as a control. Baculovirus-produced HAPR8 ectodomain possesses agglutination capability. This functional HA assay is used in conjunction with an agglutination inhibition assay for evaluation of the formulation candidates. If the HA protein or protein subdomain tested possesses hemagglutination activity before formulation, the HA-PEA-NTA-Ni⁺² vaccine must also possess hemagglutination activity.

### EXAMPLE 11

Manipulation of the Nucleic Acid Binding Capacity of NP in PEA-NTA Formulations

Bacterial expression genes were engineered to include no nucleotide sequences of ACA in the expressed mRNA to allow co-expression of the specific RNase, MazF, that targets this sequence (Suzuki, M., et al. Mol. Cell. (2005) 18:253-261). Co-induction of MazF and expression vectors for HA, M2e-NA, or NP proteins results in a lower complexity of bacterial proteins in relationship to the desired influenza proteins. This approach can both improve yield and diminish the level of bacterial proteins co-purifying with the desired influenza protein. However, in addition, the manipulation of the nucleic acids expressed at the time of promoter induction to produce the NP polypeptide enriches the inclusion of certain nucleic acids bound to a histidine-tagged NP as part of a single formulation or as part of a formulation consisting of other target antigens.

This use of a nucleic acid-binding protein as a carrier for nucleic acid is not limited to use of NP or to influenza vaccine compositions. Destruction of unwanted RNA or plasmid sequences in a cell could be selectively performed by other RNases, DNases or other targeting enzymes. Nucleic acids could be carried by other nucleic acid-binding proteins than influenza NP, including nucleic-acid binding proteins from mammalian cells, other viruses, parasites, or bacteria.

### EXAMPLE 12

Mouse Experiment

To test the effect on immunogenicity of conjugating the influenza HA and NP proteins to the invention polymer-NTP-Ni⁺²-antigen vaccine delivery compositions, 6-8 week old mice as described above were injected (day 0) with one of the following: PBS (negative control), a PEA-NTA-Ni+2 vaccine delivery composition (Example 5) either HA-PEA, NP-PEA or HA-PEA+NP-PEA and the corresponding free proteins (i.e., not conjugated to PEA SEQ ID NOS:11 and 15) or free PR8 influenza A virus as a positive control (mice injected intraperitoneally (ip) with PR8) were compared for immunoreactivity. The PBS group consisted of 10 mice, the PR8 group consisted of 3 mice, and all the other groups consisted of 5 mice each.

Animals were bled on day 20 (to assess the primary response) and boosted on day 21 with the same formulations used for priming. Animals were bled again on day 35 (to assess the secondary response) and challenged with infectious PR8 virus intranasally on day 42.

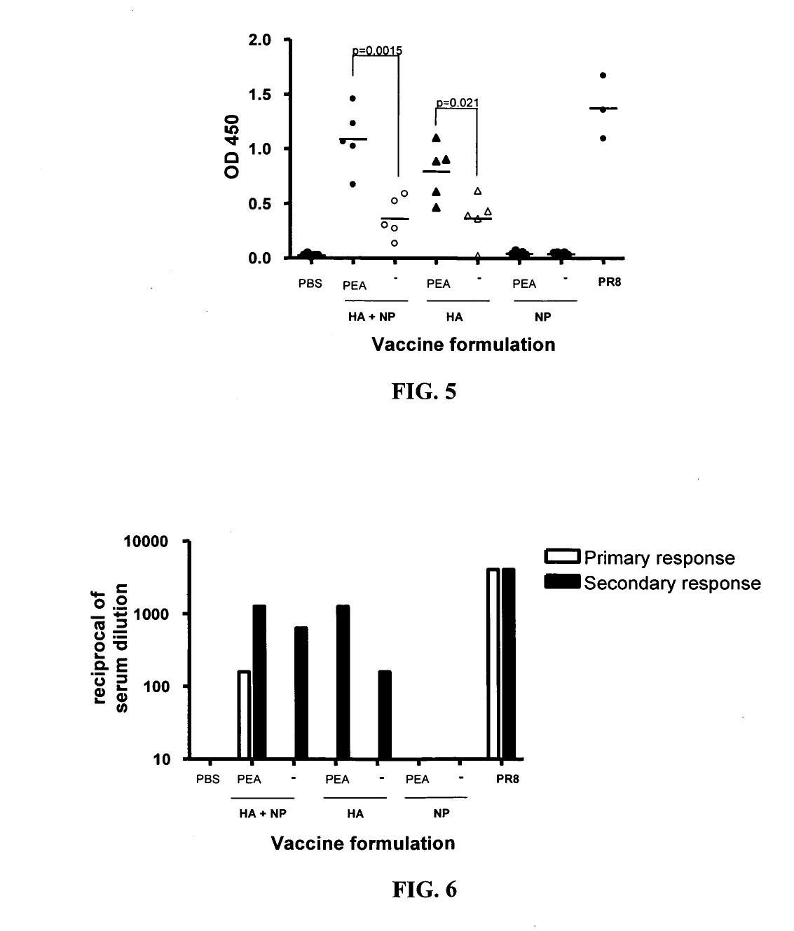

FIG. 4 summarizes the anti-HA titers from the primary antibody response for the various groups of mice. The PEA-HA+PEA-NP] vaccine induced the highest anti-HA IgG1 titer, equivalent to 8.27+/−1.39 μg of antibody per ml of serum. This titer was significantly higher (p<0.0001) than the titer induced by HA+NP injected as free proteins: 1.56+/−1.36 9 μg/ml. The antibody titer induced by the PEA-HA+PEA-NP complex was significantly higher (p=0.0056) than that induced by the PEA-HA complex: 3.92+/−2.18 μg/ml. This result indicates that the PEA-NP complex produces an immunogenic adjuvant effect. Interestingly, this adjuvant effect could only be detected when NP was delivered complexed with the PEA polymer since there was no significant difference in anti-HA titers between the PEA-HA complex (1.54+/−1.6 μg/ml) and the free HA+NP antigens (1.56+/−1.36 μg/ml). The strong adjuvant effect of the presence of the PEA polymer in the vaccine composition was also apparent in the secondary response (FIG. 5); the anti-HA IgG2a serum antibody level induced by PEA-HA+PEA-NP complex was significantly higher (p=0.0015) than the response induced by free HA+free NP. Similarly, the serum antibody level induced by PEA-HA complex was higher than that for free HA (p=0.021). The anti-HA IgG1 levels followed the same pattern of antibody titer levels and were about 100 fold higher (30-300) than the levels obtained after a single injection (see Table 4).

An essential characteristic of a preventive vaccine is its ability to quickly induce virus-neutralizing antibodies. As shown by the data summarized in FIG. 6, besides live virus, the only formulation capable of inducing neutralizing antibodies after a single injection was the PEA-HA+PEA-NP complex. By contrast, after the boost, all formulations that included HA induced measurable levels of neutralizing antibodies (FIG. 6) as measured in a microneutralization assay (Rowe, T., et al. Detection of antibody to avian influenza A (H₅N1) virus in human serum by using a combination of serologic assays. J Clin Microbiol. (1999) 37:937-43).

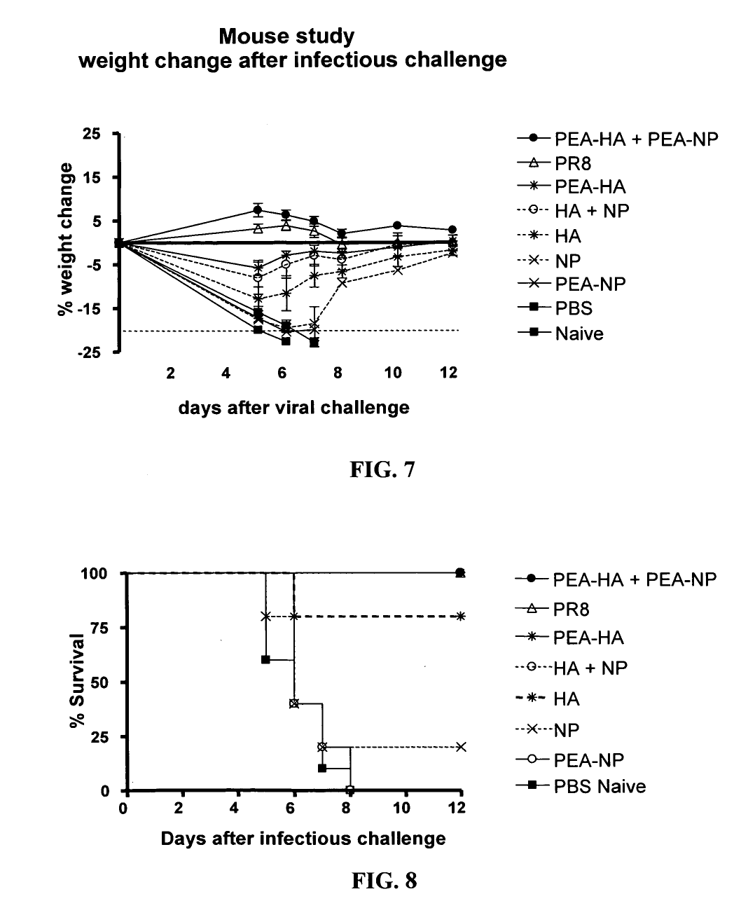

The relevance of these findings was clearly seen when the mice in this study were challenged with infectious PR8 virus. As shown in FIG. 7, which shows weight loss in study mice, the only animals that did not lose any weight up to day 4 (in fact, they kept gaining weight) were animals in the PR8-immunized group and the group injected with PEA-HA+PEA-NP complex; in all other groups, animals quickly lost weight. Importantly, animals immunized with free HA+NP or PEA-HA complex were less protected (p=0.0017 and p=0.17, respectively) than animals immunized with PEA-HA+PEA-NP complex, confirming the strong adjuvant effect of conjugation of the antigen(s) to the polymer carrier in the invention vaccine delivery composition(s) of the addition of PEA-NP complex to animals injected with PEA-HA complex. As seen from the results in FIG. 8, all animals in the naïve/PBS group and 4 out of 5 animals in the free NP group (one in the NP group had to be euthanized according to protocol), when weight loss reached 20% of the original weight. One animal in the free HA group had to be euthanized, while 100% survival was achieved in animals injected with PEA-HA+PEA-NP, PEA-HA, free HA+NP and PR8-injected groups.

TABLE 4

| PEA-HA + PEA-NP | | PEA-HA | | NP + HA | | HA | | PR8 | |
|---|---|---|---|---|---|---|---|---|---|
| d20 | d35 | d20 | d35 | d20 | d35 | d20 | d35 | d20 | d35 |
| 6.21 | 294 | 5.79 | 174 | 0.35 | 50.65 | 0.77 | 125.99 | 3.77 | 6.62 |
| 8.46 | 292 | 6.05 | 767 | 3.8 | 454 | 1.38 | 42.42 | 7.5 | 10.93 |
| 8.16 | 741 | 2.06 | 367 | 1.79 | 252 | 0 | 0.41 | 8.74 | 18.56 |
| 10.13 | 301 | 4.47 | 261 | 1 | 78.44 | 1.31 | 129.91 | | |
| 8.39 | 931 | 1.23 | 225 | 0.84 | 245 | 4.23 | 74.43 | | |

Data is reported as mg of anti-HA immunoglobulin per mL of serum using an anti-HA IgG1 monoclonal antibody as reference.

In summary, non-covalent conjugation of influenza HA to PEA produced a strong immunogen that was further improved by the addition of PEA-NP, resulting in a vaccine that prevented death and totally protected the test animals from the morbidity associated with influenza virus infection.

### EXAMPLE 13

Mouse Experiment with Influenza A/Vietnam/1203/2004 Protein Formulations

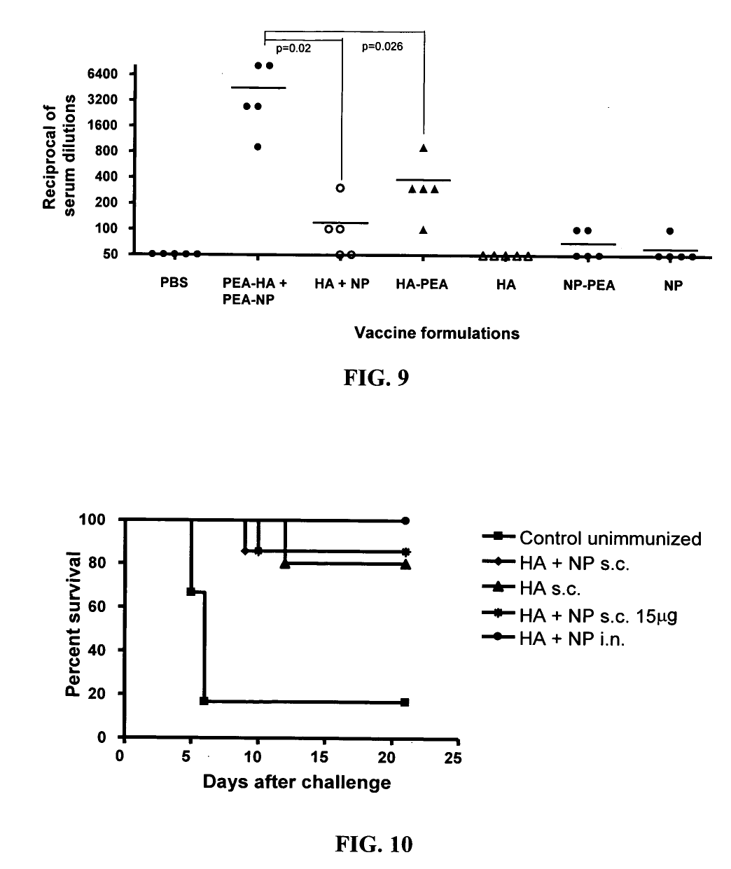

To confirm that the results obtained with PR8 can be extended to other Influenza protein subtypes, groups of 6-8 week old mice were injected (day 0) with PBS; polymer complexed proteins obtained from Influenza A/Vietnam/1203/2004—PEA-HA, PEA-NP, or PEA-HA plus PEA-NP; or the corresponding unconjugated viral proteins-HA, NP or HA+NP (SEQ ID NOS: 14 and 16). Each group consisted of 5 mice. Animals were bled 20 days later and the level of IgG1 determined by end-point ELISA. FIG. 9 represents the serum anti-HA IgG1 titers measured as the reciprocal of the dilution of serum giving an optical density (OD) reading 2 standard deviations above background. As observed in the response to HA-PR8, the PEA-HA+PEA-NP complexes based on the Vietnam influenza virus induced the highest anti-HA IgG1 titer, equivalent to 4500+/−1506 reciprocal of the serum dilution giving a positive reading. This titer was significantly higher (p<0.02) than the titer induced by free HA+NP proteins—120+/−46.4 reciprocal of the serum dilution, indicating a positive result. The combined PEA-HA+PEA-NP polymer complex was significantly more immunogenic (p=0.026) than the PEA-HA complex 380+/−135.6 reciprocal of the serum dilution, giving a positive reading. These results indicate an adjuvant effect of PEA-NP.

The results obtained in this study using vaccine compositions containing PEA polymer complexed with viral proteins derived from Influenza A/Vietnam/1203/2004 corroborate the data obtained with the proteins from the A/Puerto Rico/8/34 influenza virus.

### EXAMPLE 14

Ferret Study

Given the positive data obtained in the mouse study, the effectiveness of the invention vaccine formulations for protection conferred against A/Vietnam/1203/2004 infection in ferrets was conducted. Ferrets are considered the best model for the human influenza virus infection. Protein-polymer vaccines comprising HA and NP (SEQ ID NOS: 14 and 16), conjugated to Ni-loaded NTA-PEA were tested in ferrets at two concentrations (15 and 50 μg/ferret of the indicated protein(s)) using a prime and boost regimen, and the vaccines were tested for subcutaneous (s.c.) and intranasal (i.n.) administration. This study, performed on a contract basis at the Medical Research and Evaluation Facility of Battelle Memorial Institute (Columbus, Ohio), evaluated morbidity and mortality of the virus-challenged ferrets.

The study used 8-15 week male ferrets that were seronegative for current circulating influenza A strains. Animals were divided into five groups: Group 1) Control unimmunized (6 ferrets); Group 2) PEA-HA plus PEA-NP 50 μg subcutaneously (s.c.) (7 ferrets); Group 3) HA-PEA 50 μg, (sc) (5 ferrets); Group 4) PEA-HA plus PEA-NP 15 μg, (s.c.) (7 ferrets); and Group 5) PEA-HA plus PEA-NP 50 μg intranasally (i.n.) (6 ferrets). Ferrets in Group 4, the 15 μg group, were primed at day 0, boosted at day 28, and boosted for a second time on day 42. Ferrets in the other 3 groups were injected for the first time at day 28 and boosted on day 42. All ferrets were challenged intranasally with 1.3×10³ TCID₅₀ of A/Vietnam/1203/2004 influenza virus on day 67 of the study. Serum samples were collected throughout the study. Ferrets were observed for 20 days after challenge.

FIG. 10 shows the Kaplan and Meier survival curve for the ferrets in this study. In the PBS group, five of the six animals died. Two animals were found dead 5 days after challenge and 3 animals were euthanized 6 days after challenge because of severe neurological complications. One animal died 9 days after challenge in the PEA-HA+PEA-NP (sc) 50 μg group. One ferret died 12 days after challenge in the PEA-HA group and one ferret died 10 days after challenge in the PEA-HA+PEA-NP (sc) 15 μg group. All ferrets survived in the PEA-HA

+PEA-NP intranasal 50 μg group.

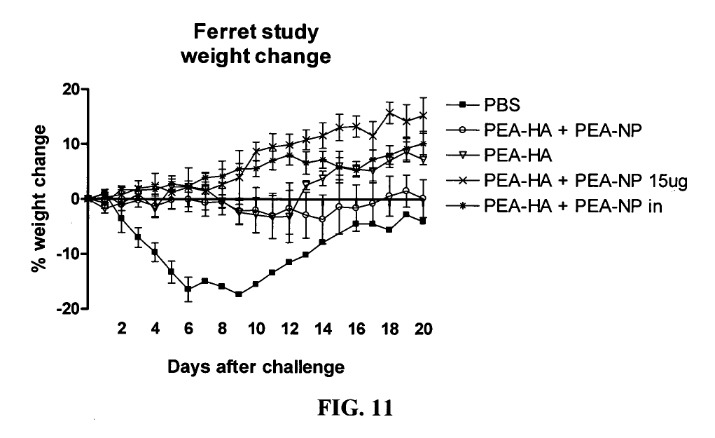

FIG. 11 is a graph showing weight changes in the study ferrets after challenge. All animals in the control group exhibited rapid weight loss, including an animal that despite losing 17% of its original weight, survived. In all other groups, ferrets reacted to the challenge well and, excluding the animals that died (see FIG. 10), lost little or no weight. In fact, many animals kept gaining weight during the entire course of the study.

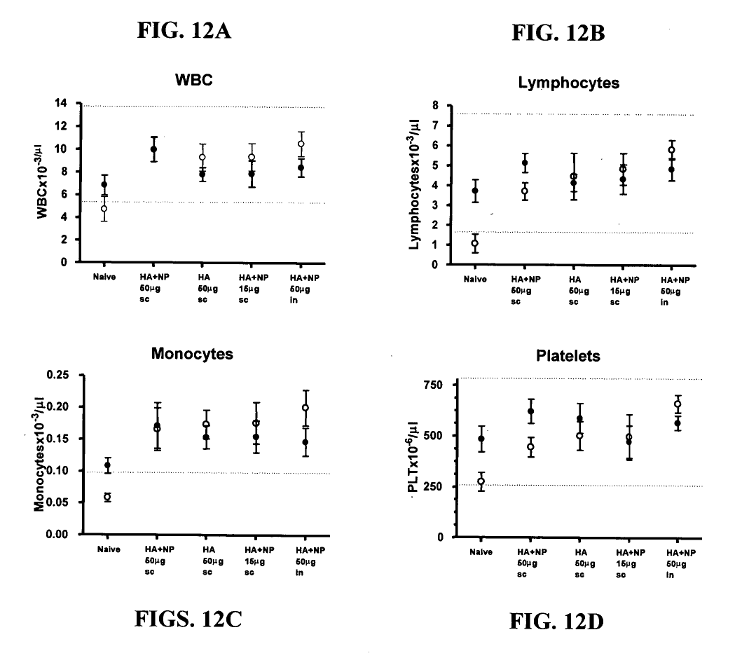

Hematological data collected from blood drawn 3 days after the infectious challenge, confirmed the lack of morbidity after infectious challenge as measured by weight loss. FIGS. 12A-D show cell counts for total white blood cells (WBC), lymphocytes, monocytes, and platelets (PLT) in the virus challenged ferrets. There was a marked reduction in all these parameters in the unimmunized group of ferrets. In contrast, the immunized animals maintained cell counts within normal ranges. This result is consistent with hematological observations of human H₅N1 patients in Vietnam (N. Engl. J. Med. (2004) 350:1179), who exhibited a severe drop in platelet count and a marked lymphopenia as prominent clinical features of their influenza infection.

In summary, based upon the results obtained in the ferret study, it can be concluded that invention anti-H₅N1 vaccine delivery compositions are effective in preventing morbidity and mortality from lethal strains of influenza A virus.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A method for assembling a polymer-based composition for delivery of a therapeutic biologic, comprising:
a) contacting together in a solution or dispersion the following elements:
1) at least one purified synthetic molecule comprising a therapeutic biologic and metal-binding amino acids;
2) at least one transition metal ion;
3) an affinity ligand that binds specifically to the metal-binding residues in the purified molecule; and
3) a synthetic biodegradable polymer containing free functional groups to which the affinity ligand can attach,
wherein the contacting is under conditions such that the affinity ligand binds to the free functional groups of the polymer and a non-covalent affinity complex forms between the transitional metal ion, the polymer-attached metal affinity ligand and the metal-binding proteins of the synthetic molecule to assemble the composition while maintaining substantial native activity for the biologic.

2. The method of claim 1, wherein the at least one transition metal ion selected from comprise a transition metal ion selected from Cu⁺, Ni²⁺, Co²⁺, and Zn²⁺ ions.

3. The method of claim 2, wherein the metal affinity ligand is selected from 6-amino-2-(bis-carboxymethylamino)-hexanoic acid, nitrilotriacetic acid (NTA), and iminodiacetic acid (IDA) and the transition metal ion is selected from Fe²⁺, Cu²⁺, or Ni²⁺.

4. The method of claim 1, wherein the metal affinity ligand is NTA and the transition metal ion is Ni²⁺.

5. The method of claim 1, wherein the metal affinity ligand and the transition metal ion are attached to the functional group of the polymer prior to the contacting in a) to assemble the composition.

6. The method of claim 1, wherein the therapeutic biologic is DNA, RNA, protein, peptide, branched peptide glycopeptide, lipopeptide, or glycolipopeptide.

7. The method of claim 5, wherein the polymer is an amino acid-containing biodegradable polymer and the free functional groups are amino or carboxyl groups.

8. The method of claim 1, wherein the biodegradable polymer comprises at least one or a blend of the following:
a poly(ester amide) (PEA) having a chemical structure described by structural formula (I) comprising from 5 to about 30 amino acids and a biodegradable PEA having a structural formula described by structural formula (I), 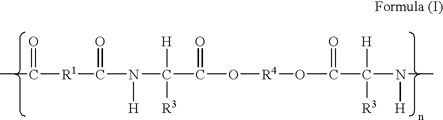
wherein n ranges from about 5 to about 150; R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof, (C₂-C₂₀) alkylene, and (C₂-C₂₀) alkenylene; 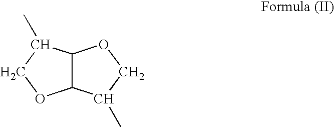
or a PEA polymer having a chemical formula described by structural formula III: 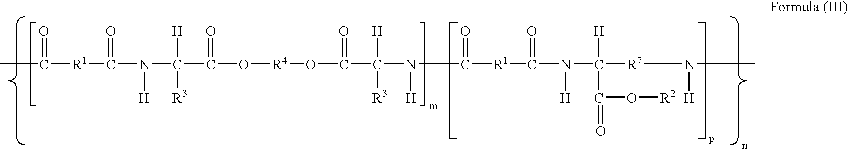
wherein n ranges from about 5 to about 150, m ranges about 0.1 to 0.9:p ranges from about 0.9 to 0.1; wherein R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol or bicyclic-fragments of 1,4:3,6-dianhydrohexitols of structural formula(II), and combinations thereof; and R⁷ is independently (C₁-C₂₀) alkyl or (C₂-C₂₀) alkenyl;
or a poly(ester urethane) (PEUR) polymer having a chemical formula described by structural formula (IV), 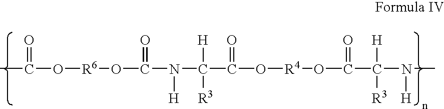
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)_{2SCH3}; R⁴ is selected from the group consisting of (C₂-C₂₀) alkylene, (C₂-C₂₀) alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀) alkylene, (C₂-C₂₀) alkenylene or alkyloxy, bicyclic-fragments of 1,4:3,6-dianhydrohexitols of general formula (II), and combinations thereof;
or a PEUR polymer having a chemical structure described by general structural formula (V) 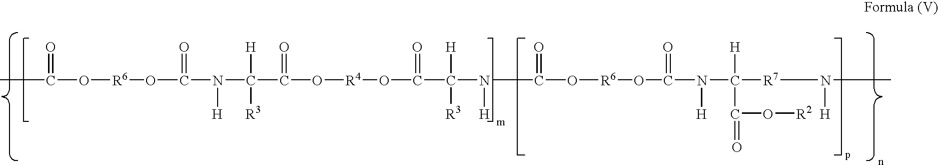
wherein n ranges from about 5 to about 150, m ranges about 0.1 to about 0.9:p ranges from about 0.9 to about 0.1; R² is independently selected from hydrogen, (C₆-C₁₀) aryl (C₁-C₂₀)alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II) and combinations thereof; and R⁶ is independently selected from (C₂-C₂₀) alkylene, (C₂-C₂₀) alkenylene or alkyloxy, bicyclic-fragments of 1,4:3,6-dianhydrohexitols of general formula (II), an effective amount of a residue of a saturated or unsaturated therapeutic diol, and combinations thereof; and R⁷ is independently (C₁-C₂₀) alkyl or (C₂-C₂₀) alkenyl
or a poly(ester urea) (PEU) having a chemical formula described by general structural formula (VI): 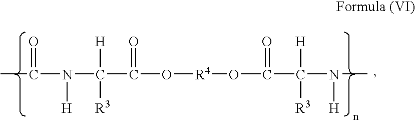
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II);
or a PEU having a chemical formula described by structural formula (VII) 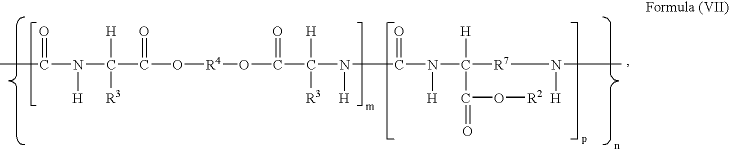
wherein m is about 0.1 to about 1.0; p is about 0.9 to about 0.1; n is about 10 to about 150; each R² is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; a bicyclic-fragment of a 1,4:3,6-dianhydrohexitol of structural formula (II), and combinations thereof;

9. The method of claim 8, wherein the polymer comprises a PEA described by structural formula (I) or (III).

10. The method of claim 8, wherein the polymer comprises a PEUR described by structural formula (IV) or (V).

11. The method of claim 8, wherein the polymer comprises a PEU described by structural formula (VI) or (VII).

12. The method of claim 8, further comprising forming particles of the polymer prior to contacting the elements together in a) to assemble the composition.

13. A method for assembling a vaccine delivery composition comprising:
a) contacting together in a solution or dispersion the following elements:
1) at least one purified molecule containing a synthetic antigen;
2) an affinity ligand that binds specifically to the purified molecule; and
3) a synthetic biodegradable polymer containing free functional groups to which the affinity ligand can be attached,
wherein the contacting is under conditions such that the affinity ligand binds to the free functional groups of the polymer and the affinity ligand forms a non-covalent complex with the molecule containing a synthetic antigen to assemble the composition.

14. The method of claim 13, wherein the polymer is an amino acid-containing biodegradable polymer and the free functional groups are amino or carboxyl groups.

15. The method of claim 13, wherein the polymer comprises at least one amino acid conjugated to at least one non-amino acid moiety per monomer.

16. The method of claim 13, wherein the biodegradable polymer comprises at least one or a blend of the following:
a poly(ester amide) (PEA) having a chemical structure described by structural formula (I) comprising from 5 to about 30 amino acids and a biodegradable PEA having a structural formula described by structural formula (I), 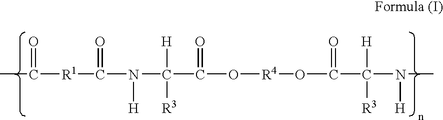
wherein n ranges from about 5 to about 150; R¹ is independently selected from residues of α,ω-bis(4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof, (C₂-C₂₀) alkylene, and (C₂-C₂₀) alkenylene; 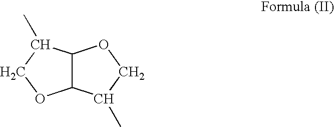
or a PEA polymer having a chemical formula described by structural formula III: 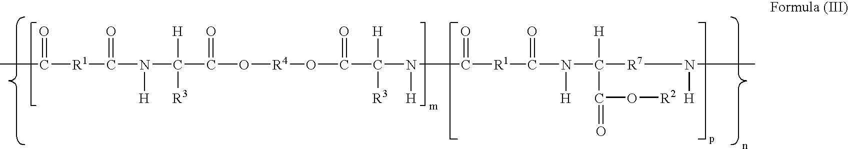
wherein n ranges from about 5 to about 150, m ranges about 0.1 to 0.9:p ranges from about 0.9 to 0.1; wherein R¹ is independently selected from residues of α,ω-bis(4-carboxyphenoxy)-(C—C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; each R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol or bicyclic-fragments of 1,4:3,6-dianhydrohexitols of structural formula(II), and combinations thereof; and R⁷ is independently (C₁-C₂₀) alkyl or (C₂-C₂₀) alkenyl;
or a poly(ester urethane) (PEUR) polymer having a chemical formula described by structural formula (IV), 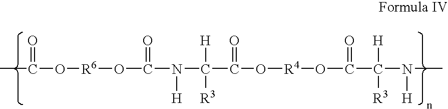
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)_{2SCH3}; R⁴ is selected from the group consisting of (C₂-C₂₀) alkylene, (C₂-C₂₀) alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀) alkylene, (C₂-C₂₀) alkenylene or alkyloxy, bicyclic-fragments of 1,4:3,6-dianhydrohexitols of general formula (II), and combinations thereof;
or a PEUR polymer having a chemical structure described by general structural formula (V) 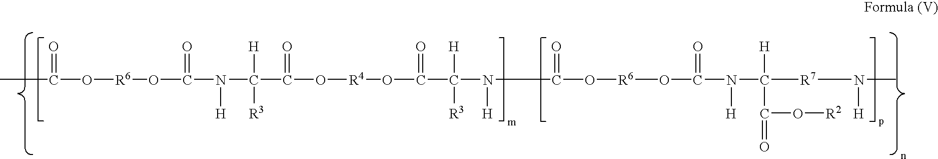
wherein n ranges from about 5 to about 150, m ranges about 0.1 to about 0.9:p ranges from about 0.9 to about 0.1; R² is independently selected from hydrogen, (C₆-C₁₀) aryl (C₁-C₂₀) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II) and combinations thereof; and
R⁶ is independently selected from (C₂-C₂₀) alkylene, (C₂-C₂₀) alkenylene or alkyloxy, bicyclic-fragments of 1,4:3,6-dianhydrohexitols of general formula (II), an effective amount of a residue of a saturated or unsaturated therapeutic diol, and combinations thereof; and R⁷ is independently (C₁-C₂₀) alkyl or (C₂-C₂₀) alkenyl or a poly(ester urea) (PEU) having a chemical formula described by general structural formula (VI): 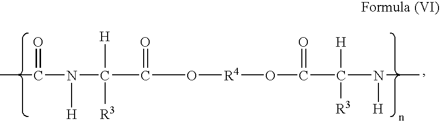
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II);
or a PEU having a chemical formula described by structural formula (VII) 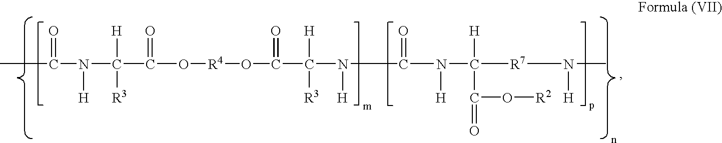
wherein m is about 0.1 to about 1.0; p is about 0.9 to about 0.1; n is about 10 to about 150; each R² is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; each R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; a bicyclic-fragment of a 1,4:3,6-dianhydrohexitol of structural formula (II), and combinations thereof;

17. The method of claim 14, wherein the polymer comprises a PEA described by structural formula (I) or (III).

18. The method of claim 14, wherein the polymer comprises a PEUR described by structural formula (IV) or (V).

19. The method of claim 14, wherein the polymer comprises a PEU described by structural formula (VI) or (VII).

20. The method of claim 14, further comprising forming particles of the polymer prior to contacting the elements together in a) to assemble the composition.

21. The method of claim 18, wherein the method further comprises forming a polymer covering on the particles.

22. The method of claim 18, wherein the particles having an average diameter in the range from about 10 nanometers to about 1000 microns and the antigen is dispersed in polymer molecules of the particles.

23. The method of claim 18, wherein the method further comprises forming a polymer covering on the particles.

24. The method of claim 18, wherein the particles have an average diameter in the range from about 10 nanometers to about 10 microns.

25. The method of claim 18, wherein a polymer molecule has an average molecular weight in a range from about 5,000 to about 300,000.

26. The method of claim 18, wherein a polymer molecule has from about 5 to about 70 antigens non-covalently attached thereto.

27. The method of claim 13, further comprising:
b) separating the complex from other elements in the solution or dispersion to purify the assembled composition.

28. The method of claim 13, wherein the complex is removed from the solution or dispersion by size-filtration.

29. The method of claim 13, further comprising binding the affinity ligand to the free functional groups of the polymer prior to contacting the elements together in a) to assemble the composition.

30. The method of claim 13, further comprising obtaining the purified molecule from a lysate or extract of an organism that contains at least one recombinant vector comprising a vector and a DNA sequence insert that encodes the synthetic antigen.

31. The method of claim 30, wherein the synthetic antigen comprises at least one Class I or Class II antigen comprising from 5 to about 30 amino acids, wherein the antigen has been expressed by the organism.

32. The method of claim 13, wherein the affinity ligand comprises a monoclonal antibody that binds specifically to the purified molecule or the synthetic antigen contained therein.

33. The method of claim 13, wherein the affinity ligand is a monoclonal antibody that binds specifically to the synthetic antigen.

34. The method of claim 33, further comprising, prior to contacting the elements together in a) to assemble the composition, conjugating the monoclonal antibody to the polymer via an antibody-binding protein domain that is bound to the polymer.

35. The method of claim 34, wherein the antibody-binding protein domain is obtained from protein A or protein G.

36. The method of claim 13, wherein the affinity ligand is a metal affinity ligand, the purified molecule comprises metal-binding amino acids, and the elements contacted together in a) further comprise a transition metal ion selected from Cu²⁺, Ni²⁺, Co²⁺, and Zn²⁺ ions.

37. The method of claim 36, wherein the metal affinity ligand is selected from 6-amino-2-(bis-carboxymethylamino)-hexanoic acid, nitrilotriacetic acid (NTA), and iminodiacetic acid (IDA) and the transition metal ion is selected from Fe²⁺, Cu²⁺, or Ni²⁺.

38. The method of claim 36, wherein the conditions comprise a pH value of about 8.

39. The method of claim 36, wherein the conditions comprise a concentration of NaCl in the range from about 0.1 M to about 1.0 M.

40. The method of claim 36, wherein the conditions comprise a concentration of NaCl in the range from about 0.5 M to about 0.9 M.

41. The method of claim 36, wherein the metal affinity ligand is NTA and the metal ion is Ni²⁺.

42. The method of claim 36, wherein the purified molecule further comprises a hexaHis tag attached to the synthetic antigen.

43. The method of claim 36, further comprising attaching the metal affinity ligand and the metal ion to the free functional groups of the polymer prior to contacting the elements together in a) to assemble the composition.

44. The method of claim 42, wherein the composition comprises from about 5 to about 150 antigens per polymer molecule.

45. The method of claim 42, further comprising forming particles of the polymer prior to contacting the elements together in a) to assemble the composition.

46. The method of claim 45, wherein the particles having an average diameter in the range from about 10 nanometers to about 1000 microns and the antigen is dispersed in polymer molecules of the particles.

47. The method of claim 36, wherein the elements contacted together in a) further comprise a peptidic adjuvant, which non-covalently binds to the polymer via a second metal affinity complex comprising the metal affinity ligand, and the metal ion.

48. The method of claim 36, wherein the elements contacted together in a) further comprise a polynucleotide adjuvant, which non-covalently binds to the polymer via a second metal affinity complex comprising the metal affinity ligand, and the metal ion.

49. The method of claim 48, wherein the elements contacted together in a) further comprise one or more Toll Like Receptor agonists.

50. The method of claim 49, wherein the elements contacted together in a) further comprise polyI:C and/or CpG.

51. The method of claim 30, wherein the DNA sequence insert further encodes one or two His tags, each having one to ten adjacent histidine residues linked to the synthetic antigen at the amino- or carboxyl-terminus thereof to encode a fusion protein.

52. The method of claim 51, wherein a single hexaHis tag is encoded at the carboxyl-terminus of the fusion protein.

53. The method of claim 30, wherein the antigen comprises a Class I or Class II antigen derived from either the H1N1 strain or the H5N1 strain of Influenza A.

54. The method of claim 53, wherein the antigen comprises an amino acid sequence as set forth in SEQ ID NO:11, 12, 13 or 14.

55. The method of claim 53, wherein the sequences derived from H5N1 of Influenza A are selected from SEQ ID NO:12, 14, 16, and combinations thereof.

56. The method of claim 13, wherein the synthetic antigen comprises a tumor-associated sugar or lipid molecule.

57. The method of claim 13, wherein the synthetic antigen comprises an epitope of a virus, bacterium, fungus or tumor cell surface antigen.

58. The method of claim 13, wherein the synthetic antigen comprises an adjuvant-binding protein or adjuvant-complexed lipo- or glyco-protein.

59. The method of claim 58, wherein the synthetic antigen comprises NP of influenza virus.

60. The method of claim 58, wherein the adjuvant is a native or synthetic polynucleotide.

61. The method of claim 60, wherein the adjuvant is one or more native or synthetic TLR agonists.

62. The method of claim 61, wherein the adjuvant is polyI:C and/or CpG.

63. The method of claim 1, wherein the composition forms a time release polymer depot when administered in vivo.

64. The method of claim 1, further comprising lyophilizing the composition.

65. A method for inducing an immune response in a mammal, said method comprising: administering to the mammal an immunostimulating amount of a vaccine delivery composition formed by the method of claim 13 in the form of a liquid dispersion of polymer particles or molecules, to induce an immune response in the mammal.

66. The method of claim 65, wherein the composition forms a time release polymer depot when administered in vivo.

67. The method of claim 65, wherein the composition biodegrades over a period of about twenty-four hours to about ninety days.

68. The method of claim 65, wherein the composition is in the form of particles having an average diameter in the range from about 10 nanometers to about 1000 microns.

69. A composition comprising a synthetic biodegradable polymer having one or more functional groups to which is preattached a metal affinity ligand that has been non-covalently complexed with a transition metal ion, wherein the composition is soluble.

70. A delivery composition made by the method of claim 1.

71. A vaccine delivery composition made by the method of claim 13.
